SEQUENCE LISTING (6 sequences)

SEQ ID NO: 1 - DNA, 1823 bp, Homo sapiens
  CDS (4)..(1110)
aaaatggtccttctgttgatcctgtcagtcttacttttgaaagaagatgtccgtgggagtgcacagtccagtgagaggagggtggtggctcacatgctgggtgacatcattattggagctctcttttctgttcatcaccagcctactgtggacgaagttcatgagaggaagtgtggggcagtccgtgaacagtatggcattcagagagtggaggccatgctgcataccctggaaaggatcaattcagaccccacactcttgcccaacatcacactgggctgtgagataagggattcctgctggcattcggctgtggccctagagcagagcattgagttcataagagattccctcatttcttcggaagaggaagagggcttggtatgctctgtggatggctcctcctcttccttccgctccaagaagcccatagtaggggtcattgggcctggttccagttctttagccattcaggtccagaatttgctccagcttttcaacatacctcagattgcttactcagcaaccatcatggatctgagtgacaagactctgttcaaatatttcatgagggttgtgccttcagatgctcagcaggcaaggtccatggtggacatagtgaagaggtacaactggacctatgtatcagccgtacacacagaaggcaactatggagaaagtgggatggaagccttcaaagatatgtcagcgaaggaagggatttgcatcgcccactcttacaaaatctacagtaatgcaggggagcagagctttgataagctgctgaagaagctcacaagtcacttgcccaaggcccgggtggtggcctacttctgtgagggcatgacggtgagaggtctgctgatggccatgaggcgcctgggtctagtgggagaatttctgcttctgggcagggaaccagatgccatctttattgagatctcaaagaacagcatcctatgggaagacagaagaaaatgccaaggtcgcttccttcagggttttggagacatattacacagaagtgagtccgtgctgctgcacatgccccagcctctgaatctagagctcagttcagggcccatcactggactgagggacaggctcatctaattctgagtggatattactctgcattataatgaagccaacagtcatatcttctgatgtggagatttgagaagcatttgtattggatgtgaccgtcaaaatgcgccccatatcactgcaacacctacaagttttcttgcatggggtgctcagactttcacctctggcaagtattactgggaggtccatgtgggggactcttggaattgggctttcggtgtttgtaataagtactggaaagggaagaatcagaatggcaatatatatggagaggagggactctttagtcttgggattgttaagaacgacattcagtgcagtctctttaccacctccccagttacactgcagtatgtcccaagacctaccaaccatgtaggattattcctggattgtgaagctagaactgtgagcttcgttgatgttaatcaaagctcccctatatacaccatccctaattgctccttctcacctcctctcaggcctatcttttgctgtattcatctctgaccagagacaaatcagaaatgtgtttatctgctgtgggaacccctttatcccataaagccctcttccttgtgccttatcaaacaggacaaataggttctgttttatgtcttgaattgcattctaatgttattaaaactcatttattgtgttactattaaatgtggtaaaamcacaaaaaaaaaaaaaaaaaaaaaaaaaaaaaa

SEQ ID NO: 2 - protein, 369 aa, Homo sapiens
MVLLLILSVLLLKEDVRGSAQSSERRVVAHMLGDIIIGALFSVHHQPTVDEVHERKCGAVREQYGIQRVEAMLHTLERINSDPTLLPNITLGCEIRDSCWHSAVALEQSIEFIRDSLISSEEEEGLVCSVDGSSSSFRSKKPIVGVIGPGSSSLAIQVQNLLQLFNIPQIAYSATIMDLSDKTLFKYFMRVVPSDAQQARSMVDIVKRYNWTYVSAVHTEGNYGESGMEAFKDMSAKEGICIAHSYKIYSNAGEQSFDKLLKKLTSHLPKARVVAYFCEGMTVRGLLMAMRRLGLVGEFLLLGREPDAIFIEISKNSILWEDRRKCQGRFLQGFGDILHRSESVLLHMPQPLNLELSSGPITGLRDRLI

SEQ ID NO: 3 - DNA, 1110 bp, Homo sapiens
  CDS (1)..(1110)
atggtccttctgttgatcctgtcagtcttacttttgaaagaagatgtccgtgggagtgcacagtccagtgagaggagggtggtggctcacatgctgggtgacatcattattggagctctcttttctgttcatcaccagcctactgtggacgaagttcatgagaggaagtgtggggcagtccgtgaacagtatggcattcagagagtggaggccatgctgcataccctggaaaggatcaattcagaccccacactcttgcccaacatcacactgggctgtgagataagggattcctgctggcattcggctgtggccctagagcagagcattgagttcataagagattccctcatttcttcggaagaggaagagggcttggtatgctctgtggatggctcctcctcttccttccgctccaagaagcccatagtaggggtcattgggcctggttccagttctttagccattcaggtccagaatttgctccagcttttcaacatacctcagattgcttactcagcaaccatcatggatctgagtgacaagactctgttcaaatatttcatgagggttgtgccttcagatgctcagcaggcaaggtccatggtggacatagtgaagaggtacaactggacctatgtatcagccgtacacacagaaggcaactatggagaaagtgggatggaagccttcaaagatatgtcagcgaaggaagggatttgcatcgcccactcttacaaaatctacagtaatgcaggggagcagagctttgataagctgctgaagaagctcacaagtcacttgcccaaggcccgggtggtggcctacttctgtgagggcatgacggtgagaggtctgctgatggccatgaggcgcctgggtctagtgggagaatttctgcttctgggcagggaaccagatgccatctttattgagatctcaaagaacagcatcctatgggaagacagaagaaaatgccaaggtcgcttccttcagggttttggagacatattacacagaagtgagtccgtgctgctgcacatgccccagcctctgaatctagagctcagttcagggcccatcactggactgagggacaggctcatctaa

SEQ ID NO: 4 - protein, 1212 aa, Homo sapiens
MVLLLILSVLLLKEDVRGSAQSSERRVVAHMPGDIIIGALFSVHHQPTVDKVHERKCGAVREQYGIQRVEAMLHTLERINSDPTLLPNITLGCEIRDSCWHSAVALEQSIEFIRDSLISSEEEEGLVRCVDGSSSSFRSKKPIVGVIGPGSSSVAIQVQNLLQLFNIPQIAYSATSMDLSDKTLFKYFMRVVPSDAQQARAMVDIVKRYNWTYVSAVHTEGNYGESGMEAFKDMSAKEGICIAHSYKIYSNAGEQSFDKLLKKLTSHLPKARVVACFCEGMTVRGLLMAMRRLGLAGEFLLLGSDGWADRYDVTDGYQREAVGGITIKLQSPDVKWFDDYYLKLRPETNHRNPWFQEFWQHRFQCRLEGFPQENSKYNKTCNSSLTLKTHHVQDSKMGFVINAIYSMAYGLHNMQMSLCPGYAGLCDAMKPIDGRKLLESLMKTNFTGVSGDTILFDENGDSPGRYEIMNFKEMGKDYFDYINVGSWDNGELKMDDDEVWSKKSNIIRSVCSEPCEKGQIKVIRKGEVSCCWTCTPCKENEYVFDEYTCKACQLGSWPTDDLTGCDLIPVQYLRWGDPEPIAAVVFACLGLLATLFVTVVFIIYRDTPVVKSSSRELCYIILAGICLGYLCTFCLIAKPKQIYCYLQRIGIGLSPAMSYSALVTKTNRIARILAGSKKKICTKKPRFMSACAQLVIAFILICIQLGIIVALFIMEPPDIMHDYPSIREVYLICNTTNLGVVTPLGYNGLLILSCTFYAFKTRNVPANFNEAKYIAFTMYTTCIIWLAFVPIYFGSNYKIITMCFSVSLSATVALGCMFVPKVYIILAKPERNVRSAFTTSTVVRMHVGDGKSSSAASRSSSLVNLWKRRGSSGETLRYKDRRLAQHKSEIECFTPKGSMGNGGRATMSSSNGKSVTWAQNEKSSRGQHLWQRLSIHINKKENPNQTAVIKPFPKSTESRGLGAGAGAGGSAGGVGATGGAGCAGAGPGGPESPDAGPKALYDVAEAEEHFPAPARPRSPSPISTLSHRAGSASRTDDDVPSLHSEPVARSSSSQGSLMEQISSVVTRFTANISELNSMMLSTAAPSPGVGAPLCSSYLIPKEIQLPTTMTTFAEIQPLPAIEVTGGAQPAAGAQAAGDAARESPAAGPEAAAAKPDLEELVALTPPSPFRDSVDSGSTTPNSPVSESALCIPSSPKYDTLIIRDYTQSSSSL

SEQ ID NO: 5 - protein, 1203 aa, Rattus sp.
MVLLLILSVLLLKEDVRGSAQSSERRVVAHMPGDIIIGALFSVHHQPTVDKVHERKCGAVREQYGIQRVEAMLHTLERINSDPTLLPNITLGCEIRDSCWHSAVALEQSIEFIRDSLISSEEEEGLVRCVDGSSSFRSKKPIVGVIGPGSSSVAIQVQNLLQLFNIPQIAYSATSMDLSDKTLFKYFMRVVPSDAQQARAMVDIVKRYNWTYVSAVHTEGNYGESGMEAFKDMSAKEGICIAHSYKIYSNAGEQSFDKLLKKLRSHLPKARVVACFCEGMTVRGLLMAMRRLGLAGEFLLLGSDGWADRYDVTDGYQREAVGGITIKLQSPDVKWFDDYYLKLRPETNLRNPWFQEFWQHRFQCRLEGFAQENSKYNKTCNSSLTLRTHHVQDSKMGFVINAIYSMAYGLHNMQMSLCPGYAGLCDAMKPIDGRKLLDSLMKTNFTGVSGDMILFDENGDSPGRYEIMNFKEMGKDYFDYINVGSWDNGELKMDDDEVWSKKNNIIRSVCSEPCEKGQIKVIRKGEVSCCWTCTPCKENEYVFDEYTCKACQLGSWPTDDLTGCDLIPVQYLRWGDPEPIAAVVFACLGLLATLFVTVIFIIYRDTPVVKSSSRELCYIILAGICLGYLCTFCLIAKPKQIYCYLQRIGIGLSPAMSYSALVTKTNRIARILAGSKKKICTKKPRFMSACAQLVIAFILICIQLGIIVALFIMEPPDIMHDYPSIREVYLICNTTNLGVVTPLGYNGLLILSCTFYAFKTRNVPANFNEAKYIAFTMYTTCIIWLAFVPIYFGSNYKIITMCFSVSLSATVALGCMFVPKVYIILAKPERNVRSAFTTSTVVRMHVGDGKSSSAASRSSSLVNLWKRRGSSGETLRYKDRRLAQHKSEIECFTPKGSMGNGGRATMSSSNGKSVTWAQNEKSTRGQHLWQRLSVHINKKENPNQTAVIKPFPKSTENRGPGAAAGGGSGPGVAGAGNAGCTATGGPEPPDAGPKALYDVAEAEESFPAAARPRSPSPISTLSHLAGSAGRTDDDAPSLHSETAARSSSSQGSLMEQISSVVTRFTANISELNSMMLSTAATPGPPGTPICSSYLIPKEIQLPTTMTTFAEIQPLPAIEVTGGAQGATGVSPAQETPTGAESAPGKPDLEELVALTPPSPFRDSVDSGSTTPNSPVSESALCIPSSPKYDTLIIRDYTQSSSSL

SEQ ID NO: 6 - protein, 19 aa, Artificial Sequence
  misc_feature 1: Leu or Val
XXNXXXLFXIXQXXXXXXX

(19) United States
(12) Patent Application Publication
(10) Pub. No.: US 2002/0142330 A1
(43) Pub. Date: Oct. 3, 2002

(54) Novel glutamate receptor modulatory proteins and nucleic acid molecules and uses therefor

(75) Inventors: Brian Gaither Bates, Chelmsford, MA (US); Yuhong Xie, Brookline, MA (US); Kamalakar Gulukota, Lawrenceville, NJ (US); Janet Elizabeth Paulsen, Londonderry, NH (US)

Correspondence Address: WYETH, FIVE GIRALDA FARMS, MADISON, NJ, 07940, US

(73) Assignee: Genetics Institute, Inc., Cambridge, MA

(21) Appl. No.: 10/027,923

(22) Filed: Dec. 21, 2001

Related U.S. Application Data
(60) Provisional application No. 60/257,589, filed on Dec. 22, 2000.

(51) Int. Cl. C12Q 1/68; G01N 33/53; G06F 19/00; G01N 33/50; C07H 21/04; C12P 21/02; C12N 5/06; C07K 14/705

(52) U.S. Cl. 435/6; 435/69.1; 435/320.1; 435/325; 530/350; 536/23.5; 702/20

(57) ABSTRACT

Novel mGluR5M polypeptides, proteins, and nucleic acid molecules are disclosed. In addition to isolated, full-length mGluR5M proteins, the invention further provides isolated mGluR5M fusion proteins, antigenic peptides and anti-mGluR5M antibodies. The invention also provides mGluR5M nucleic acid molecules, recombinant expression vectors containing a nucleic acid molecule of the invention, host cells into which the expression vectors have been introduced and non-human transgenic animals in which a mGluR5M gene has been introduced or disrupted. Diagnostic, screening and therapeutic methods utilizing compositions of the invention are also provided.

### RELATED APPLICATIONS

This application claims the benefit of U.S. Provisional Application Serial No. 60/257,589, filed Dec. 22, 2000, the entire contents of which are incorporated herein by this reference.

### BACKGROUND OF THE INVENTION

Glutamate is the transmitter of the vast majority of the exicitatory synapses in the mammalian central nervous system (CNS) and plays an important role in a wide variety of CNS functions (for review, see Hollmann and Heinemann (1994) Annu. Rev. Neurosci. 17:31-108). The understanding of the glutamatergic synapse has advanced enormously in the last 10 years, primarily through application of molecular biological techniques to the study of glutamate receptors and transporters. There are three families of glutamate-gated cation channels termed ionotropic glutamate receptors (iGluRs); the N-methyl-D-aspartate (NMDA) receptor, the alpha-amino-3-hydroxy-5-methyl-4-isoxazolepropionic acid (AMPA) receptor and the kainate receptor. There are also three groups (based of their amino acid identity) of metabotropic, G protein-coupled glutamate receptors (mGluRs) that modify neuronal and glial excitability through G protein subunits acting on membrane ion channels and second messengers such as diacylglycerol and cAMP. In addition, there are at least two glial glutamate transporters and three neuronal transporters in the brain.

The glutamate receptors are known to be important in modulating a variety of neuronal activities including, but not limited to, neural plasticity, neural development and neurodegeneration. The NMDA receptors and AMPA/kainate receptors act as glutamate-gated cation channels, whereas metabotropic receptors (mGluRs) modulate the production of second messengers via G proteins. Molecular studies indicate that NMDA receptors exist as multiple subunits (NMDAR1 and NMDAR2A-2D) and that and mGluRs exist as multiple subtypes (mGluR1 -mGluR8). Moreover, splice variants have been found for at least three mGluRs: mGluR1, mGluR4 and mGluR5.

The subtypes mGluR1 and mGluR5 are coupled to the stimulation of the phosphatidylinositol-calcium second messenger system while mGluR2, mGluR3, mGluR4, mGluR6, mGluR7 and mGluR8 are coupled to G rpoteins that inhibit adenylate cyclase activity. The metabotropic glutamate receptors (mGluRs), for example mGluR1 and mGluR5, can increase intracellular Ca2+ concentration via Ins(1,4,5)P3- and ryanodine-sensitive Ca2+ stores in neurons. Both types of store are coupled functionally to Ca2+-permeable channels found in the plasma membrane. The mGluR-mediated increase in intracellular Ca2+ concentration can activate Ca2+-sensitive K+ channels and Ca2+-dependent nonselective cationic channels. These mGluR-mediated effects often result from mobilization of Ca2+ from ryanodine-sensitive, rather than Ins(1,4,5)P3-sensitive, Ca2+ stores, suggesting that close functional interactions exist between mGluRs, intracellular Ca2+ stores and Ca2+-sensitive ion channels in the membrane.

With respect to the metabotropic glutamate receptor subtype 5 (mGluR5) two isoforms have been identified in the rat and human brain. Compared with mGlu5a, mGlu5b contains an insertion of 32 amino acids, 50 residues after the seventh transmembrane domain. The primary sequence of the human receptors share about 93-96% idendity compared with the rat homologues. The deduced amino acid sequence of the large extracellular domain is extremely well conserved between rat and human mGluR5 (98.6%) which suggests that the amino-terminal region of mGluR5 is functionally important. Expression of either human isoform in Xenopus oocytes evokes glutamate responses, which suggests that the two receptors from the human brain could activate phospholipase C and generate Ca(2+)-activated Cl-current.

Because of the ubiquitous distribution of glutamatergic synapses, mGluRs have the potential to participate in a wide variety of functions in the CNS. In addition, the wide diversity and heterogeneous distribition of mGluR subtypes provide an opportunity for developing pharmacologic agents that selectively interact with mGluRs involved in only one or a limited number of CNS functions. Accordingly, there exists a need to gain a detailed understanding of the specific roles of mGluRs in CNS function and to identify modulators of particular mGluRs, in particular, for use in developing novel strategies for a variety of psychiatric and neurological disorders.

### SUMMARY OF THE INVENTION

The present invention is based, at least in part, on the discovery of nucleic acid molecules which encode a novel family of secreted proteins having homology to the metabotropic glutamate receptor subtype 5 subfamily, in particular, to the N-terminal portion of the metabotropic glutamate receptors mGluR5a and mGluR5b, referred to herein as the metabotropic glutamate receptor subtype 5 modulatory proteins (also referred to herein as the “mGluR5M” proteins or “mGluR5M” family). The mGluR5M molecules of the present invention as well as mGluR5M mimics and/or mGluR5M modulators, are useful in regulating a variety of cellular processes. Accordingly, in one aspect, this invention provides isolated nucleic acid molecules encoding mGluR5M proteins or biologically active portions thereof, as well as nucleic acid fragments suitable as primers or hybridization probes for the detection of mGluR5M-encoding nucleic acids.

In one embodiment, a mGluR5M nucleic acid molecule is 60% identical to the nucleotide sequence shown in SEQ ID NO:1, the nucleotide sequence of the DNA insert of the plasmid deposited with ATCC as Accession Number PTA-2775, or complement thereof. In a preferred embodiment, an isolated mGluR5M nucleic acid molecule has the nucleotide sequence shown SEQ ID NO:3, or a complement thereof. In another preferred embodiment, an isolated mGluR5M nucleic acid molecule has the nucleotide sequence shown in SEQ ID NO:1, or a complement thereof. In yet another preferred embodiment, an isolated nucleic acid molecule has the nucleotide sequence of the DNA insert of the plasmid deposited with ATCC as Accession Number PTA-2775, or a complement thereof.

In another embodiment, a mGluR5M nucleic acid molecule includes a nucleotide sequence encoding a protein having an amino acid sequence sufficiently homologous or identical to the amino acid sequence of SEQ ID NO:2 or an amino acid or an amino acid sequence encoded by the DNA insert of the plasmid deposited with the ATCC as Accession No. PTA-2775. In another preferred embodiment, a mGluR5M nucleic acid molecule includes a nucleotide sequence encoding a protein having an amino acid sequence at least 60% identical to the amino acid sequence of SEQ ID NO:2 or an amino acid or an amino acid sequence encoded by the DNA insert of the plasmid deposited with the ATCC as Accession No. PTA-2775.

In another embodiment, an isolated nucleic acid molecule of the present invention encodes a mGluR5M protein which includes an N-terminal mGluR-like domain and/or a C-terminal unique domain. In another embodiment, an isolated nucleic acid molecule of the present invention encodes a protein which includes a G protein coupled receptor family 3 consensus sequence. In yet another embodiment, a mGluR5M nucleic acid molecule encodes a mGluR5M protein which lacks a transmembrane domain. In yet another embodiment, a mGluR5M nucleic acid molecule encodes a mGluR5M protein and is a naturally occurring nucleotide sequence. In yet another embodiment, a mGluR5M nucleic acid molecule encodes a mGluR5M protein which lacks a transmembrane domain.

Another embodiment of the invention features mGluR5M nucleic acid molecules which specifically detect mGluR5M nucleic acid molecules relative to nucleic acid molecules encoding non-mGluR5M proteins. For example, in one embodiment, a mGluR5M nucleic acid molecule is at least 30 nucleotides in length and hybridizes under stringent conditions to a complement of a nucleic acid molecule comprising the nucleotide sequence shown in SEQ ID NO:1 or the nucleotide sequence of the DNA insert of the plasmid deposited with ATCC as Accession Number PTA-2775. In another embodiment, a mGluR5M nucleic acid molecule hybridizes under stringent conditions to a complement of a nucleic acid molecule consisting of about nucleotides 880-1823 of SEQ ID NO:1. Another embodiment of the invention provides an isolated nucleic acid molecule which is antisense to the coding strand of a mGluR5M nucleic acid.

Another aspect of the invention provides a vector comprising a mGluR5M nucleic acid molecule. In certain embodiments, the vector is a recombinant expression vector. In another embodiment, the invention provides a host cell containing a vector of the invention. The invention also provides a method for producing a mGluR5M protein by culturing in a suitable medium, a host cell of the invention containing a recombinant expression vector such that a mGluR5M protein is produced.

Another aspect of this invention features isolated or recombinant mGluR5M proteins and polypeptides. In one embodiment, an isolated mGluR5M protein includes an N-terminal mGluR-like domain. In another embodiment, an isolated mGluR5M protein includes a C-terminal unique domain. In another embodiment, an isolated mGluR5M protein lacks a transmembrane domain. In another embodiment, an isolated mGluR5M protein has an amino acid sequence sufficiently homologous or identical to the amino acid sequence of SEQ ID NO:2. In a preferred embodiment, a mGluR5M protein has an amino acid sequence at least about 60% identical to the amino acid sequence of SEQ ID NO:2. In another embodiment, a mGluR5M protein has the amino acid sequence of SEQ ID NO:2.

Another embodiment of the invention features an isolated mGluR5M protein which is encoded by a nucleic acid molecule having a nucleotide sequence at least about 60%, 65%, 70%, 75%, 80%, 85%, 90%, 91%, 92%, 93%, 94%, 95%, 96%, 97%, 98%, 99% or more identical to a nucleotide sequence of SEQ ID NO:1, or a complement thereof. Yet another embodiment of the invention features an isolated mGluR5M protein which is at least about 60%, 65%, 70%, 75%, 80%, 85%, 90%, 91%, 92%, 93%, 94%, 95%, 96%, 97%, 98%, 99% or more identical to a polypeptide having the amino acid sequence of SEQ ID NO:2. This invention further features an isolated mGluR5M protein which is encoded by a nucleic acid molecule having a nucleotide sequence which hybridizes under stringent hybridization conditions to a complement of a nucleic acid molecule comprising the nucleotide sequence of SEQ ID NO:1.

The mGluR5M proteins of the present invention, or biologically active portions thereof, can be operatively linked to a non-mGluR5M polypeptide to form mGluR5M fusion proteins. The invention further features antibodies that specifically bind mGluR5M proteins, such as monoclonal or polyclonal antibodies. In addition, the mGluR5M proteins or biologically active portions thereof can be incorporated into pharmaceutical compositions, which optionally include pharmaceutically acceptable carriers.

In another aspect, the present invention provides a method for detecting mGluR5M expression in a biological sample by contacting the biological sample with an agent capable of detecting a mGluR5M nucleic acid molecule, protein or polypeptide such that the presence of a mGluR5M nucleic acid molecule, protein or polypeptide is detected in the biological sample.

In another aspect, the present invention provides a method for detecting the presence of mGluR5M activity in a biological sample by contacting the biological sample with an agent capable of detecting an indicator of mGluR5M activity such that the presence of mGluR5M activity is detected in the biological sample.

In another aspect, the invention provides a method for modulating mGluR5 activity comprising contacting the cell with an mGluR5M protein, peptide, nucleic acid molecule, peptidomimetic or other small molecule such that mGluR5 activity in the cell is modulated. In one embodiment, the mGluR5M protein, peptide, antibody, nucleic acid molecule, peptidomimetic or other small molecule inhibits mGluR5 activity. In another embodiment, the mGluR5M protein, peptide, antibody, nucleic acid molecule, peptidomimetic or other small molecule stimulates mGluR5 activity. In a preferred embodiment, the agent is an antibody that specifically binds to a mGluR5M protein. In another embodiment, the agent is a mGluR5M nucleic acid molecule. In yet another embodiment, the agent is a mGluR5M protein, peptide of peptidomimetic.

In one embodiment, the methods of the present invention are used to treat a subject having a disorder characterized by aberrant mGluR expression or activity by administering a mGluR5M protein, peptide, antibody, nucleic acid molecule, peptidomimetic or other small molecule to the subject. In a preferred embodiment, the disorder characterized by aberrant mGluR5M protein or nucleic acid expression is central nervous system disorder.

The present invention also provides a diagnostic assay for identifying the presence or absence of a genetic alteration characterized by at least one of (i) aberrant modification or mutation of a gene encoding a mGluR5M protein; (ii) mis-regulation of said gene; and (iii) aberrant post-translational modification of a mGluR5M protein, wherein a wild-type form of said gene encodes an protein with a mGluR5M activity.

In another aspect the invention provides a method for identifying a compound that binds to or modulates the activity of a mGluR5M protein. In one embodiment, the invention provides a method for identifying a compound which binds to a mGluR5M protein which involves contacting the mGluR5M protein with a test compound (optionally in addition to mGluR5 ligand) and determining whether the mGluR5M protein binds to the test compound. In another embodiment, the invention provides a method for identifying a compound which binds to or modulates the activity of a mGluR5M protein which involves contacting a cell expressing a mGluR5 protein with a mGluR5M protein and a test compound, and determining the effect of the test compound on the binding or activity of the mGluR5M protein to thereby identify a compound which modulates the activity of the mGluR5M protein.

Other features and advantages of the invention will be apparent from the following detailed description and claims.

### BRIEF DESCRIPTION OF THE DRAWINGS

FIG. 1 depicts the cDNA sequence and predicted amino acid sequence of human mGluR5M. The nucleotide sequence (panel A) corresponds to nucleic acids 1 to 1823 of SEQ ID NO:1. The coding region is underlined. The amino acid sequence (panel B) corresponds to amino acids 1 to 369 of SEQ ID NO:2.

FIG. 2 depicts a multiple sequence alignment (MSA) of the amino acid sequences of human mGluR5M (SEQ ID NO:2), the first 370 amino acid residues of human mGluR5 (amino acid residues 1 to 370 of SEQ ID NO:4), and the first 369 amino acid residues of rat mGluR5 (amino acid residues 1 to 369 of SEQ ID NO:5). The alignment was performed using the Clustal algorithm which is part of the MEGALIGN program (e.g., version 3.1.7) which is part of the DNASTAR sequence analysis software package. The pairwise alignment parameters are as follows: K-tuple=1; Gap Penalty=3; Window=5; Diagonals saved=5 . The multiple alignment parameters are as follows: Gap Penalty=10; and Gap length penalty=10. The asterisks indicate important conserved residues in proteins aligned.

### DETAILED DESCRIPTION OF THE INVENTION

The present invention is based on the discovery of novel molecules, referred to herein as metabotropic glutamate receptor subtype 5 modulatory (“mGluR5M”) protein and nucleic acid molecules, which comprise a family of molecules having certain conserved structural and functional features. The term “family” when referring to the protein and nucleic acid molecules of the invention is intended to mean two or more proteins or nucleic acid molecules having a common structural domain and having sufficient amino acid or nucleotide sequence homology or identity as defined herein. Such family members can be naturally occurring and can be from either the same or different species. For example, a family can contain a first protein of human origin, as well as other, distinct proteins of human origin or alternatively, can contain homologues of non-human origin. Members of a family may also have common functional characteristics.

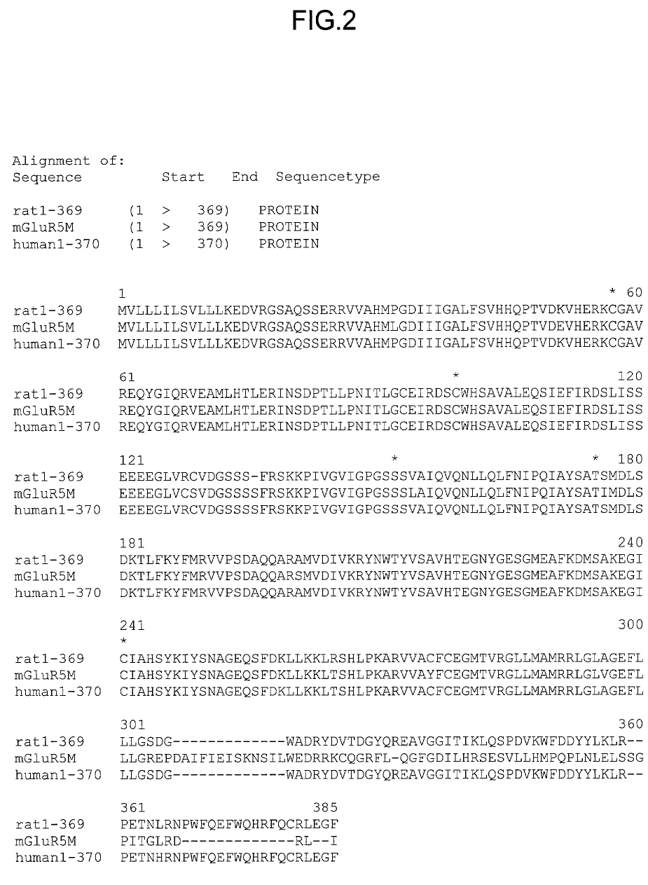

For example, the mGluR5M proteins of the present invention bear significant homology to the N-terminal extracellular domain of the metabotropic glutamate receptors, mGluR5a and mGluR5b. The metabotropic glutamte receptors are known to share certain conserved amino acid residues, some of which have been determined to be important in ligand binding and/or receptor function. For example, the metabotropic glutamate receptors are known to share at least 19 conserved cysteine residues within the N-terminal extracellular domain and extracellular loops which are proposed to be important in the three dimensional structure of the receptors and/or in intramolecular transduction. At least three of these conserved cysteine residues are present in the human mGluR5M amino acid sequence set forth as SEQ ID NO:2. Moreover, based on homology of the N-terminal extracellular domains of the metabotropic glutamate receptors to known bacterial periplasmic binding proteins (PBLs), a serine residue (located at amino acid 152 in the human mGluR5 sequence set forth as SEQ ID NO:4) and a threonine residue (located at at amino acid 175 in the human mGluR5 sequence set forth as SEQ ID NO:4) are predicted to be important in glutamate binding, both of which are present in the human mGluR5M amino acid sequence set forth as SEQ ID NO:2 (i.e., Ser152 and Thr175 of SEQ ID NO:2). These important conserved residues are indicated by asterisks in FIG. 2.

In one aspect, the mGluR5M proteins of the present invention are proteins having an amino acid sequence of about 330-410, preferably about 340-400, more preferably about 350-390, more preferably about 360-380, or about 369-370 amino acids in length, and include at least one domain or consensus sequence characteristic of the family of mGluR5M family, as described herein. In another embodiment, the mGluR5M proteins of the present invention contain a signal sequence. As used herein, a “signal sequence” includes a peptide of about 20 amino acid residues in length which occurs at the N-terminus of secretory and integral membrane proteins and which contains at least 30% hydrophobic amino acid residues. In a preferred embodiment, a signal sequence contains at least about 15, 20, 25, 30, 35, 40 or more amino acid residues and has at least about 30%, 35%, 40%, 45%, 50%, 55%, 60% or more hydrophobic amino acid residues (e.g., Alanine, Valine, Leucine, Isoleucine, Phenylalanine, Tyrosine, Tryptophan, or Proline). Such a “signal sequence”, also referred to in the art as a “signal peptide”, serves to direct a protein containing such a sequence to a lipid bilayer. For example, a signal sequence can be found about amino acids 1-20 of SEQ ID NO:2 (Met1 to Ala20 of the mGluR5M amino acid sequence set forth as SEQ ID NO:2). In yet another embodiment, a mGluR5M protein is a mature mGluR5M protein. As used herein, the term “mature mGluR5M protein” refers to a mGluR5M protein from which the signal peptide has been cleaved. In an exemplary embodiment, a mature mGluR5M protein contains amino acid residues 21-369 of SEQ ID NO:2.

In another embodiment, a mGluR5M protein of the present invention includes an N-terminal mGluR-like domain. As used herein, the term “N-terminal mGluR-like domain” refers to a protein domain having an amino acid sequence of about 250-350 amino acids which shares at least about 80% identity with a mGluR amino acid sequence. Preferably, an “N-terminal mGluR-like domain” includes a protein domain having an amino acid sequence of about 260-340, 270-330, 280-320, 290-310 or about 300 amino acid residues (e.g., about 302 amino acid residues) and shares at least about 85%, 90%, 91%, 92%, 93%, 94%, 95%, 96%, 97%, 98%, 99% or more identity with a mGluR amino acid sequence. In an exemplary embodiment, a mGluR5M protein contains an N-terminal mGluR-like domain including about amino acids 1-303 of SEQ ID NO:2.

In yet another embodiment, a mGluR5M protein of the present invention includes a C-terminal unique domain. The term “C-terminal unique domain” as used herein, refers to a protein domain of a mGluR5M protein family member which includes amino acid residues C-terminal to the N-terminal mGluR-like domain in the amino acid sequence of the mGluR5M protein. As used herein, a “C-terminal unique domain” refers to a protein domain which is at about 50-75 amino acid residues in length, preferably at least about 55-70 amino acid residues in length, and more preferably at least about 60-65 amino acid residues in length (e.g., about 63 amino acid residues in length), and has at least about 60%, 65%, 70%, 75%, 80%, 85%, 90%, 91%, 92%, 93%, 94%, 95%, 96%, 97%, 98%, 99% or more identity with the C-terminal amino acid sequence of another mGluR5M family member. As further defined herein, a C-terminal unique domain of a mGluR5M protein family member, however, is not sufficiently homologous to the amino acid sequence of a mGluR. For example, a C-terminal unique domain of mGluR5M homologue has at least about 80% homology to the C-terminal unique domain of human mGluR5M from about amino acids 304-369 of SEQ ID NO:2 but has no significant homology to the amino acid sequence of human mGluR5.

A mGluR5M family member (or an N-terminal mGluR-like domain) can further be identified based on the presence of at least one G-protein coupled receptor family 3 signature consensus sequence. For example, a mGluR5M protein can include a G-protein coupled receptor family 3_{—}1 consensus having the sequence [LV]-X-N-[LIVM](2)-X-L-F-X-I-[PA]-Q-[LIVM]-[STA]-X-[STA](3)-[STAN] (SEQ ID NO:6). The consensus sequence described herein is described according to standard Prosite Signature designation (e.g., all amino acids are indicated according to their universal single letter designation; X designates any amino acid; X(n) designates any n amino acids, e.g., X (2) designates any 2 amino acids; and [LIVM] indicates any one of the amino acids appearing within the brackets, e.g., any one of L, I, V, or M, in the alternative, any one of Leu, Ile, Val, or Met.)

In yet another embodiment, a mGluR5M protein of the present invention lacks a transmembrane domain. As used herein, a “transmembrane domain” includes a protein domain having at least about 10 amino acid residues of which about 60% of the amino acid residues contain non-polar side chains, for example, alanine, valine, leucine, isoleucine, proline, phenylalanine, tryptophan, and methionine. In a preferred embodiment, a “transmembrane domain” includes a protein domain having at least about 13, preferably about 16, more preferably about 19, and even more preferably about 21, 23, 25, 30, 35 or 40 amino acid residues, of which at least about 70%, preferably about 80%, and more preferably about 90% of the amino acid residues contain non-polar side chains, for example, alanine, valine, leucine, isoleucine, proline, phenylalanine, tryptophan, and methionine. A transmembrane domain is lipophillic in nature, and spans a membrane of a cell (e.g., spans the cell membrane or an organelle membrane in a eukaryotic cell). Proteins or polypeptides having a transmembrane domain (or transmembrane domains) are commonly referred to as “membrane-bound” proteins or polypeptides. A protein or polypeptide that “lacks a transmembrane domain” is conversly, not membrane-bound, for example is cytosolic, secreted or exists within an organelle-millieu.

Preferred mGluR5M molecules of the present invention have an amino acid sequence sufficiently homologous or identical to the mGluR5M protein having the amino acid sequence of SEQ ID NO:2. As used herein, the term “sufficiently homologous” or “sufficiently identical” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains and/or a common functional activity. For example, amino acid or nucleotide sequences which share common structural domains share at least about 60%, 65%, 70%, 75%, 80%, 85%, 90%, 91%, 92%, 93%, 94%, 95%, 96%, 97%, 98%, 99% or more identity across the amino acid sequences of the domains and contain at least one and preferably two structural domains, are defined herein as sufficiently homologous or identical. Furthermore, amino acid or nucleotide sequences which share at least about 60%, 65%, 70%, 75%, 80%, 85%, 90%, 91%, 92%, 93%, 94%, 95%, 96%, 97%, 98%, 99% or more identity and share a common functional activity are defined herein as sufficiently homologous or identical.

As used interchangeably herein, a “mGluR5M activity”, “biological activity of mGluR5M” or “functional activity of mGluR5M”, refers to an activity exerted by a mGluR5M protein, polypeptide or nucleic acid molecule, for example, an activity exerted on a mGluR-expressing cell, as determined in vivo, or in vitro, according to standard techniques.

In a preferred aspect on the invention, a “mGluR5M activity”, “biological activity of mGluR5M” or “functional activity of mGluR5M” includes modulation (e.g., enhancement or inhibition) of glutamate receptor functions and/or activities, in particular, metabotropic glutamate receptor functions and/or activities (e.g., mGluR5 functions and/or activities). In one embodiment, a mGluR5M protein of the present invention modulates glutamate receptor function and/or activity directly, for example, by binding to, enhancing or inhibiting receptor activity in a ligand dependent or independent manner. In another embodiment, a mGluR5M protein of the present invention modulates glutamate receptor function and/or activity indirectly, e.g., by influencing glutamate receptor ligand availability. For example, a mGluR5M protein can regulate neurotransmission, neurotoxicity, exitotoxicity and/or metabolic function by direct binding of glutamate.

In a preferred embodiment, a mGluR5M activity is at least one of the following activities: (1) modulation of G protein linked second messenger signaling pathways (e.g., modulation of diacylglycerol and/or inisitol triphosphate-mediated signaling pathways), for example, signaling pathways involved in neuronal cell signalling and nervous system function; (2) modulation of glutamatergic transmission; (3) modulation of neuronal excitability; (4) regulation of synaptic transmission; (5) modulation of neurotransmitter release (e.g., glutamate release); (6) regulation of voltage-dependent and/or voltage-independent and/or ligand-gated ion channels (e.g., K+channels or Ca2+ channels); (7) regulation of neuronal development (e.g., regulation of neuronal differentiation, migration and/or survival in the developing brain); (8) modulatoin of nervous system function; and (9) modulation of neurodegenerative processes (e.g., acute or chronic neurodegenerative processes). In yet another embodiment, a mGluR5M activity is modulation of mGluR5 dimerization (e.g., mGluR5a and/or mGluR5b dimerization) and/or dimerization of other mGluR family members (e.g., mGluR1 dimerization).

Accordingly, the mGluR5M proteins of the present invention (as well as peptides, nucleic acid molecules, antibodies, peptidomimetics and small molecules) may be useful in treating, for example, neurodegenerative disorders and/or diseases (e.g., motor neuron disease (MND), amyotrophic lateral sclerosis (ALS), Huntington's chorea, Parkinson's disease and Alzheimer's disease), stoke, the brain damage occurring acutely after status epilepticus, cerebral ischemia or traumatic brain injury and/or movement disorders. The mGluR5M proteins of the present invention (as well as peptides, nucleic acid molecules, antibodies, peptidomimetics and small molecules) may also be useful as cognitive enhancers, antiepileptic agents and/or in the treatment of pain and/or hypertension. Other clinical conditions that may respond to treatment with the mGluR5M proteins of the present invention include epilepsy, amnesia, anxiety, hyperalgesia and/or psychiatric disorders (e.g., schizophrenia and/or other psychoses). In a preferred, the mGluR5M proteins of the present invention (as well as peptides, nucleic acid molecules, antibodies, peptidomimetics and small molecule modulators) are useful in the treatment of shizophrenia and/or other psychiatric disorders including, but not limited to schizoaffective disorder, bipolar affective disorder, unipolar affective disorder and adolescent conduct disorder.

A preferred embodiment of the invention features isolated mGluR5M proteins and polypeptides having a mGluR5M activity, as described herein. Preferred mGluR5M proteins have at least an N-terminal mGluR-like domain and/or a C-terminal unique domain and a mGluR5M activity. In a preferred embodiment, a mGluR5M protein has a G-protein coupled receptor family 3_{—}1 consensus sequence and a mGluR5M activity. In another preferred embodiment, a mGluR5M protein has at least an N-terminal mGluR-like domain and/or a C-terminal unique domain and a G-protein coupled receptor family 3_{—}1 consensus sequence, a mGluR5M activity, and an amino acid sequence sufficiently homologous or identical to the amino acid sequence of SEQ ID NO:2.

A human mGluR5M cDNA was identified from a library derived from human adult whole brain RNA, which is approximately 1823 nucleotides in length, and which encodes a protein which is approximately 369 amino acid residues in length. The human mGluR5M protein contains an N-terminal mGluR-like domain at about amino acids 1 to 303 of SEQ ID NO:2 and contains a C-terminal unique domain at about amino acids 304 to 369 of SEQ ID NO:2. The human mGluR5M protein further contains a G-protein coupled receptor family 3_{—}1 consensus seqeunce at about amino acids 158 to 176 of SEQ ID NO:2.

A plasmid containing the nucleotide sequence encoding human mGlu5M (referred to interchangeably herein as YI176) was deposited with the American Type Culture Collection (ATCC), 10801 University Boulevard, Manassas, VA 20110-2209, on Dec. 12, 2000 and assigned Accession Number PTA-2775. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

Various aspects of the invention are described in further detail in the following subsections:

I. Isolated Nucleic Acid Molecules

One aspect of the invention pertains to isolated nucleic acid molecules that encode mGluR5M proteins or biologically active portions thereof, as well as nucleic acid fragments sufficient for use as hybridization probes to identify mGluR5M-encoding nucleic acids (e.g., mGluR5M mRNA) and fragments for use as PCR primers for the amplification or mutation of mGluR5M nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated mGluR5M nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:1, the nucleotide sequence of the DNA insert of the plasmid deposited with ATCC as Accession Number PTA-2775, or a portion thereof, can be isolated using standard molecular biology techniques and the sequence information provided herein. Using all or portion of the nucleic acid sequence of SEQ ID NO:1, or the nucleotide sequence of the DNA insert of the plasmid deposited with ATCC as Accession Number PTA-2775 as a hybridization probe, mGluR5M nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1, or the nucleotide sequence of the DNA insert of the plasmid deposited with ATCC as Accession Number PTA-2775 can be isolated by the polymerase chain reaction (PCR) using synthetic oligonucleotide primers designed based upon the sequence of SEQ ID NO:1, or the nucleotide sequence of the DNA insert of the plasmid deposited with ATCC as Accession Number PTA-2775.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to mGluR5M nucleotide sequences can be prepared by standard synthetic techniques, e.g., using an automated DNA synthesizer.

In a preferred embodiment, an isolated nucleic acid molecule of the invention comprises the nucleotide sequence shown in SEQ ID NO:1. The sequence of SEQ ID NO:1 corresponds to the human mGluR5M cDNA. This cDNA comprises sequences encoding the human mGluR5M protein (i.e., “the coding region”, from nucleotides 4-1113, including a stop codon from nucleotides 1110-1113), as well as 5′ untranslated sequences (nucleotides 1-3) and 3′ untranslated sequences (nucleotides 1110 or 1113-1823). Alternatively, the nucleic acid molecule can comprise only the coding region of SEQ ID NO:1 (e.g., nucleotides 4 to 1110 or 1113, corresponding to SEQ ID NO:3).

In another preferred embodiment, an isolated nucleic acid molecule of the invention comprises a nucleic acid molecule which is a complement of the nucleotide sequence shown in SEQ ID NO:1, the nucleotide sequence of the DNA insert of the plasmid deposited with ATCC as Accession Number PTA-2775, or a portion of any of these nucleotide sequences. A nucleic acid molecule which is complementary to the nucleotide sequence shown in SEQ ID NO:1, or the nucleotide sequence of the DNA insert of the plasmid deposited with ATCC as Accession Number PTA-2775 is one which is sufficiently complementary to the nucleotide sequence shown in SEQ ID NO:1, or the nucleotide sequence of the DNA insert of the plasmid deposited with ATCC as Accession Number PTA-2775, thereby forming a stable duplex.

In still another preferred embodiment, an isolated nucleic acid molecule of the present invention comprises a nucleotide sequence which is at least about 60%, 65%, 70%, 75%, 80%, 85%, 90%, 91%, 92%, 93%, 94%, 95%, 96%, 97%, 98%, 99% or more identical to the nucleotide sequences shown in SEQ ID NO:1, or the nucleotide sequence of the DNA insert of the plasmid deposited with ATCC as Accession Number PTA-2775, or a portion of any of these nucleotide sequences.

Moreover, the nucleic acid molecule of the invention can comprise only a portion of the nucleic acid sequence of SEQ ID NO:1, or the nucleotide sequence of the DNA insert of the plasmid deposited with ATCC as Accession Number PTA-2775, for example a fragment which can be used as a probe or primer or a fragment encoding a biologically active portion of a mGluR5M protein. The nucleotide sequence determined from the cloning of the mGluR5M genes allows for the generation of probes and primers designed for use in identifying and/or cloning other mGluR5M family members, as well as mGluR5M homologues from other species. The probe/primer typically comprises substantially purified oligonucleotide. The probe/primer (e.g., oligonucleotide) typically comprises about 5-10, 10-15, 15-20, 20-25 or more nucleotides that hybridize under stringent conditions to a sense sequence of SEQ ID NO:1 or the nucleotide sequence of the DNA insert of the plasmid deposited with ATCC as Accession Number PTA-2775, to an anti-sense sequence of SEQ ID NO:1 or the nucleotide sequence of the DNA insert of the plasmid deposited with ATCC as Accession Number PTA-2775, or to a naturally occurring mutant of SEQ ID NO:1. Alternatively, the probe/primer (e.g., oligonucleotide) comprises about 5-10, 10-15, 15-20, 20-25 or more consecutive nucleotides of a sense sequence of SEQ ID NO:1 or the nucleotide sequence of the DNA insert of the plasmid deposited with ATCC as Accession Number PTA-2775, of an anti-sense sequence of SEQ ID NO:1 or the nucleotide sequence of the DNA insert of the plasmid deposited with ATCC as Accession Number PTA-2775, or of a naturally occurring mutant of SEQ ID NO:1. An exemplary probe/primer for detection of mGluR5M seqeunces comprises nucleotides of a sense or antisense sequence of SEQ ID NO:1 from about nucleotides 880 to 1823.

Probes based on the mGluR5M nucleotide sequences can be used to detect transcripts or genomic sequences encoding the same or homologous proteins. In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a mGluR5M protein, such as by measuring a level of a mGluR5M-encoding nucleic acid in a sample of cells from a subject e.g., detecting mGluR5M mRNA levels or determining whether a genomic mGluR5M gene has been mutated or deleted.

A nucleic acid fragment encoding a “biologically active portion of a mGluR5M protein” can be prepared by isolating a portion of the nucleotide sequence of SEQ ID NO:1, or the nucleotide sequence of the DNA insert of the plasmid deposited with ATCC as Accession Number PTA-2775, which encodes a polypeptide having a mGluR5M biological activity (the biological activities of the mGluR5M proteins have previously been described), expressing the encoded portion of the mGluR5M protein (e.g., by recombinant expression in vitro) and assessing the activity of the encoded portion of the mGluR5M protein. In an exemplary embodiment, a nucleic acid molecule encoding a biologically active portion of a protein of the present invention comprises a nucleotide sequence which is greater that 100, 200, 300, 400, 500, 600, 700, 800, 900, 1000, 1100, 1200, 1300, 1400, 1500, 1600, 1700, 1800 or more nucleotides in length and hybridizes under stringent hybridization conditions to a complement of the nucleic acid molecule of SEQ ID NO:1 or the nucleotide sequence of the DNA insert of the plasmid deposited with ATCC as Accession Number PTA-2775.

The invention further encompasses nucleic acid molecules that differ from the nucleotide sequence shown in SEQ ID NO:1 or the nucleotide sequence of the DNA insert of the plasmid deposited with ATCC as Accession Number PTA-2775, due to degeneracy of the genetic code and thus encode the same mGluR5M proteins as those encoded by the nucleotide sequence shown in SEQ ID NO:1. In another embodiment, an isolated nucleic acid molecule of the invention has a nucleotide sequence encoding a protein having an amino acid sequence shown in SEQ ID NO:2.

In addition to the mGluR5M nucleotide sequences shown in SEQ ID NO:1, or the nucleotide sequence of the DNA insert of the plasmid deposited with ATCC as Accession Number PTA-2775, it will be appreciated by those skilled in the art that DNA sequence polymorphisms that lead to changes in the amino acid sequences of the mGluR5M proteins may exist within a population (e.g., the human population). Such genetic polymorphism in the mGluR5M genes may exist among individuals within a population due to natural allelic variation. As used herein, the terms “gene” and “recombinant gene” refer to nucleic acid molecules comprising an open reading frame encoding a mGluR5M protein, preferably a mammalia mGluR5M protein. Such natural allelic variations can typically result in 1-5% variance in the nucleotide sequence of a mGluR5M gene. Any and all such nucleotide variations and resulting amino acid polymorphisms in mGluR5M genes that are the result of natural allelic variation and that do not alter the functional activity of a mGluR5M protein are intended to be within the scope of the invention.

Moreover, nucleic acid molecules encoding other mGluR5M family members and thus which have a nucleotide sequence which differs from the mGluR5M sequence of SEQ ID NO:1 are intended to be within the scope of the invention. For example, a primate mGluR5M cDNA can be identified based on the nucleotide sequence of human mGluR5M. Nucleic acid molecules encoding mGluR5M proteins from different species, and thus which have a nucleotide sequence which differs from the mGluR5M sequences of SEQ ID NO:1 are intended to be within the scope of the invention.

Nucleic acid molecules corresponding to natural allelic variants and homologues of the mGluR5M cDNAs of the invention can be isolated based on their homology to the mGluR5M nucleic acids disclosed herein using the cDNAs disclosed herein, or a portion thereof, as a hybridization probe according to standard hybridization techniques under stringent hybridization conditions.

Accordingly, in another embodiment, an isolated nucleic acid molecule of the invention is at least 15 nucleotides in length and hybridizes under stringent conditions to a complement of the nucleic acid molecule comprising the nucleotide sequence of SEQ ID NO:1 or the nucleotide sequence of the DNA insert of the plasmid deposited with ATCC as Accession Number PTA-2775. In another embodiment, the nucleic acid is at least 30, 50, 100, 250 or 500 nucleotides in length and hybridizes under stringent conditions to a complement of a nucleic acid molecule comprising the nucleotide sequence of SEQ ID NO:1 or the nucleotide sequence of the DNA insert of the plasmid deposited with ATCC as Accession Number PTA-2775.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions includes hybridization in 4× sodium chloride/sodium citrate (SSC), at about 65-70° C. (or hybridization in 4× SSC plus 50% formamide at about 42-50° C.) followed by one or more washes in IX SSC, at about 65-70° C. A preferred, non-limiting example of highly stringent hybridization conditions includes hybridization in 1× SSC, at about 65-70° C. (or hybridization in IX SSC plus 50% formamide at about 42-50° C.) followed by one or more washes in 0.3× SSC, at about 65-70° C. A preferred, non-limiting example of reduced stringency hybridization conditions includes hybridization in 4× SSC, at about 50-60° C. (or alternatively hybridization in 6× SSC plus 50% formamide at about 40-45° C) followed by one or more washes in 2× SSC, at about 50-60° C. Ranges intermediate to the above-recited values, e.g., at 65-70° C. or at 42-50° C. are also intended to be encompassed by the present invention. SSPE (1× SSPE is 0.15M NaCl, 10 mM NaH₂PO₄, and 1.25 mM EDTA, pH 7.4) can be substituted for SSC (1× SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(%G+C)−(600/N), where N is the number of bases in the hybrid, and [Na⁺x] is the concentration of sodium ions in the hybridization buffer ([Na⁺] for 1× SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C., see e.g., Church and Gilbert (1984) Proc. Natl. Acad. Sci. USA 81:1991-1995, (or alternatively 0.2× SSC, 1% SDS).

Preferably, an isolated nucleic acid molecule of the invention that hybridizes under stringent conditions to a complement of the sequence of SEQ ID NO:1 or 3 and corresponds to a naturally-occurring nucleic acid molecule. As used herein, a “naturally-occurring” nucleic acid molecule refers to an RNA or DNA molecule having a nucleotide sequence that occurs in nature (e.g., encodes a natural polypeptide).

In addition to naturally-occurring allelic variants of the mGluR5M sequences that may exist in the population, the skilled artisan will further appreciate that changes can be introduced by mutation into the nucleotide sequences of SEQ ID NO:1 or the nucleotide sequence of the DNA insert of the plasmid deposited with ATCC as Accession Number PTA-2775, thereby leading to changes in the amino acid sequence of the encoded mGluR5M proteins, without altering the functional ability of the mGluR5M proteins. For example, nucleotide substitutions leading to amino acid substitutions at “non-essential” amino acid residues can be made in the sequence of SEQ ID NO:1 or the nucleotide sequence of the DNA insert of the plasmid deposited with ATCC as Accession Number PTA-2775. A “non-essential” amino acid residue is a residue that can be altered from the wild-type sequence of mGluR5M (e.g., the sequence of SEQ ID NO:2) without altering the biological activity, whereas an “essential” amino acid residue is required for biological activity. For example, amino acid residues that are conserved among the mGluR5M proteins of the present invention and mGluR5 proteins and asterisked in FIG. 2 are predicted to be particularly unamenable to alteration. Moreover, amino acid residues that are defined by the G protein coupled receptor family 3_{—}1 consensus sequence are particularly unamenable to alteration. Furthermore, additional amino acid residues that are conserved between the mGluR5M proteins of the present invention and other members of the G protein coupled receptor protein family (e.g., the mGlu5R proteins set forth in FIG. 2) are not likely to be amenable to alteration.

Accordingly, another aspect of the invention pertains to nucleic acid molecules encoding mGluR5M proteins that contain changes in amino acid residues that are not essential for activity. Such mGluR5M proteins differ in amino acid sequence from SEQ ID NO:2 yet retain biological activity. In one embodiment, the isolated nucleic acid molecule comprises a nucleotide sequence encoding a protein, wherein the protein comprises an amino acid sequence at least about 60%, 65%, 70%, 75%, 80%, 85%, 90%, 91%, 92%, 93%, 94%, 95%, 96%, 97%, 98%, 99% or more identical to SEQ ID NO:2.

An isolated nucleic acid molecule encoding a mGluR5M protein homologous to the protein of SEQ ID NO:2 can be created by introducing one or more nucleotide substitutions, additions or deletions into the nucleotide sequence of SEQ ID NO:1, such that one or more amino acid substitutions, additions or deletions are introduced into the encoded protein. Mutations can be introduced into SEQ ID NO:1 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a mGluR5M protein is preferably replaced with another amino acid residue from the same side chain family. Alternatively, in another embodiment, mutations can be introduced randomly along all or part of a mGluR5M coding sequence, such as by saturation mutagenesis, and the resultant mutants can be screened for mGluR5M biological activity to identify mutants that retain activity. Following mutagenesis of SEQ ID NO:1, or the encoded protein can be expressed recombinantly and the activity of the protein can be determined.

In a preferred embodiment, a mutant mGluR5M protein can be assayed for the ability to (1) modulate G protein linked second messenger signaling pathways (e.g., modulate diacylglycerol and/or inisitol triphosphate-mediated signaling pathways); (2) modulate glutamatergic transmission; (3) modulate neuronal excitability; (4) regulate synaptic transmission; (5) modulate neurotransmitter release (e.g., glutamate release); (6) regulate voltage-dependent and/or voltage-independent and/or ligand-gated ion channels (e.g., K+ channels or Ca2+ channels); (7) regulate neuronal development (e.g., regulate neuronal differentiation, migration and/or survival in the developing brain); (8) modulate neurodegenerative processes (e.g., acute or chronic neurodegenerative processes); and/or (9) modulate mGluR5 dimerization (e.g., mGluR5a and/or mGluR5b dimerization).

In addition to the nucleic acid molecules encoding mGluR5M proteins described above, another aspect of the invention pertains to isolated nucleic acid molecules which are antisense thereto. An “antisense” nucleic acid comprises a nucleotide sequence which is complementary to a “sense” nucleic encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire mGluR5M coding strand, or to only a portion thereof. In one embodiment, an antisense nucleic acid molecule is antisense to a “coding region” of the coding strand of a nucleotide sequence encoding mGluR5M. The term “coding region” refers to the region of the nucleotide sequence comprising codons which are translated into amino acid residues (e.g., the coding region of human mGluR5M corresponds to SEQ ID NO:3). In another embodiment, the antisense nucleic acid molecule is antisense to a “noncoding region” of the coding strand of a nucleotide sequence encoding mGluR5M. The term “noncoding region” refers to 5′ and 3′ sequences which flank the coding region that are not translated into amino acids (i.e., also referred to as 5′ and 3′ untranslated regions).

Given the coding strand sequences encoding mGluR5M disclosed herein (e.g., SEQ ID NO:3), antisense nucleic acids of the invention can be designed according to the rules of Watson and Crick base pairing. The antisense nucleic acid molecule can be complementary to the entire coding region of mGluR5M mRNA, but more preferably is an oligonucleotide which is antisense to only a portion of the coding or noncoding region of mGluR5M mRNA. For example, the antisense oligonucleotide can be complementary to the region surrounding the translation start site of mGluR5M mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5- oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a mGluR5M protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave mGluR5M mRNA transcripts to thereby inhibit translation of mGluR5M mRNA. A ribozyme having specificity for a mGluR5M-encoding nucleic acid can be designed based upon the nucleotide sequence of a mGluR5M cDNA disclosed herein (i.e., SEQ ID NO:1). For example, a derivative of a Tetrahymena L-19 IVS RNA can be constructed in which the nucleotide sequence of the active site is complementary to the nucleotide sequence to be cleaved in a mGluR5M-encoding mRNA. See, e.g., Cech et al. U.S. Pat. No. 4,987,071; and Cech et al. U.S. Pat. No. 5,116,742. Alternatively, mGluR5M mRNA can be used to select a catalytic RNA having a specific ribonuclease activity from a pool of RNA molecules. See, e.g., Bartel, D. and Szostak, J. W. (1993) Science 261:1411-1418.

Alternatively, mGluR5M gene expression can be inhibited by targeting nucleotide sequences complementary to the regulatory region of the mGluR5M (e.g., the mGluR5M promoter and/or enhancers) to form triple helical structures that prevent transcription of the mGluR5M gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

In yet another embodiment, the mGluR5M nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. PNAS 93: 14670-675.

PNAs of mGluR5M nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of mGluR5M nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In another embodiment, PNAs of mGluR5M can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of mGluR5M nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. US. 86:6553-6556; Lemaitre et al. (1987) Proc. Natl. Acad. Sci. USA 84:648-652; PCT Publication No. WO88/09810, published Dec. 15, 1988) or the blood-brain barrier (see, e.g., PCT Publication No. WO89/10134, published Apr. 25, 1988). In addition, oligonucleotides can be modified with hybridization-triggered cleavage agents (See, e.g., Krol et al. (1988) BioTechniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

II. Isolated mGluR5M Proteins and Anti-mGluR5M Antibodies

One aspect of the invention pertains to isolated mGluR5M proteins, and biologically active portions thereof, as well as polypeptide fragments suitable for use as immunogens to raise anti-mGluR5M antibodies. In one embodiment, native mGluR5M proteins can be isolated from cells or tissue sources by an appropriate purification scheme using standard protein purification techniques. In another embodiment, mGluR5M proteins are produced by recombinant DNA techniques. Alternative to recombinant expression, a mGluR5M protein or polypeptide can be synthesized chemically using standard peptide synthesis techniques.

An “isolated” or “purified” protein or biologically active portion thereof is substantially free of cellular material or other contaminating proteins from the cell or tissue source from which the mGluR5M protein is derived, or substantially free from chemical precursors or other chemicals when chemically synthesized. The language “substantially free of cellular material” includes preparations of mGluR5M protein in which the protein is separated from cellular components of the cells from which it is isolated or recombinantly produced. In one embodiment, the language “substantially free of cellular material” includes preparations of mGluR5M protein having less than about 30% (by dry weight) of non-mGluR5M protein (also referred to herein as a “contaminating protein”), more preferably less than about 20% of non-mGluR5M protein, still more preferably less than about 10% of non-mGluR5M protein, and most preferably less than about 5% non-mGluR5M protein. When the mGluR5M protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of mGluR5M protein in which the protein is separated from chemical precursors or other chemicals which are involved in the synthesis of the protein. In one embodiment, the language “substantially free of chemical precursors or other chemicals” includes preparations of mGluR5M protein having less than about 30% (by dry weight) of chemical precursors or non-mGluR5M chemicals, more preferably less than about 20% chemical precursors or non-mGluR5M chemicals, still more preferably less than about 10% chemical precursors or non-mGluR5M chemicals, and most preferably less than about 5% chemical precursors or non-mGluR5M chemicals.

Biologically active portions of a mGluR5M protein include peptides comprising amino acid sequences sufficiently homologous to or derived from the amino acid sequence of the mGluR5M protein, e.g., the amino acid sequence shown in SEQ ID NO:2, which include less amino acids than the full length mGluR5M proteins, and exhibit at least one activity of a mGluR5M protein. Typically, biologically active portions comprise a domain or motif with at least one activity of the mGluR5M protein. A biologically active portion of a mGluR5M protein can be a polypeptide which is, for example, 10, 25, 50, 100 or more amino acids in length.

In one embodiment, a biologically active portion of a mGluR5M protein comprises at least an N-terminal mGluR-like domain and/or a C-terminal unique domain. In another embodiment, a biologically active portion of a mGluR5M protein comprises at least one G protein coupled receptor family 3_{—}1 consensus sequence.

It is to be understood that a preferred biologically active portion of a mGluR5M protein of the present invention may contain at least one of the above-identified structural domains and/or profiles. A more preferred biologically active portion of a mGluR5M protein may contain at least two of the above-identified structural domains and/or profiles. Moreover, other biologically active portions, in which other regions of the protein are deleted, can be prepared by recombinant techniques and evaluated for one or more of the functional activities of a native mGluR5M protein.

In a preferred embodiment, the mGluR5M protein has an amino acid sequence shown in SEQ ID NO:2. In other embodiments, the mGluR5M protein is substantially homologous or identical to SEQ ID NO:2, and retains the functional activity of the protein of SEQ ID NO:2, yet differs in amino acid sequence due to natural allelic variation or mutagenesis, as described in detail in subsection I above. Accordingly, in another embodiment, the mGluR5M protein is a protein which comprises an amino acid sequence at least about 60% identical to the amino acid sequence of SEQ ID NO:2 and retains the functional activity of the mGluR5M proteins of SEQ ID NO:2, respectively. Preferably, the protein is at least about 65%, 70%, 75%, 80%, 85%, 90%, 91%, 92%, 93%, 94%, 95%, 96%, 97%, 98%, 99% or more identical to the amino acid sequence of SEQ ID NO:2 and retains the functional activity of the mGluR5M proteins of SEQ ID NO:2.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the mGluR5M amino acid sequence of SEQ ID NO:2 having 369 amino acid residues, at least 111, preferably at least 148, more preferably at least 185, even more preferably at least 221, and even more preferably at least 258, 295 or 332 amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at http://www.gcg.com), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A preferred, non-limiting example of parameters to be used in conjunction with the GAP program include a Blosum 62 scoring matrix with a gap penalty of 12, a gap extend penalty of 4, and a frameshift gap penalty of 5.

In another embodiment, the percent identity between two amino acid or nucleotide sequences is determined using the algorithm of E. Meyers and W. Miller (Comput. Appl. Biosci., 4:11-17 (1988)) which has been incorporated into the ALIGN program (version 2.0 or version 2.0U), using a PAM120 weight residue table, a gap length penalty of 12 and a gap penalty of 4.

The nucleic acid and polypeptide sequences of the present invention can further be used as a “query sequence” to perform a search against public databases to, for example, identify other family members or related sequences. Such searches can be performed using the NBLAST and XBLAST programs (version 2.0) of Altschul, et al. (1990) J Mol. Biol. 215:403-10. BLAST nucleotide searches can be performed with the NBLAST program, score=100, wordlength=12 to obtain nucleotide sequences homologous to mGluR5M nucleic acid molecules of the invention. BLAST protein searches can be performed with the XBLAST program, score=100, wordlength=3, and a Blosum62 matrix to obtain amino acid sequences homologous to mGluR5M polypeptide molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

Proteins and protein fragments of the present invention include proteins with amino acid sequence lengths that are at least 25% (more preferably at least 50%, and even more preferably preferably at least 75%) of the length of a disclosed protein and have at least 60-70% sequence identity (more preferably, at least 70-75% identity and even more preferably at least 75-80%, 80-85%, 85-90%, 90-95% or more identity) with that disclosed protein, where sequence identity is determined as described herein.

The invention also provides mGluR5M chimeric or fusion proteins. As used herein, a mGluR5M “chimeric protein” or “fusion protein” comprises a mGluR5M polypeptide operatively linked to a non-mGluR5M polypeptide. A “mGluR5M polypeptide” refers to a polypeptide having an amino acid sequence corresponding to mGluR5M, whereas a “non-mGluR5M polypeptide” refers to a polypeptide having an amino acid sequence corresponding to a protein which is not substantially homologous to the mGluR5M protein, e.g., a protein which is different from the mGluR5M protein and which is derived from the same or a different organism. Within a mGluR5M fusion protein the mGluR5M polypeptide can correspond to all or a portion of a mGluR5M protein. In a preferred embodiment, a mGluR5M fusion protein comprises at least one biologically active portion of a mGluR5M protein. In another preferred embodiment, a mGluR5M fusion protein comprises at least two biologically active portions of a mGluR5M protein. Within the fusion protein, the term “operatively linked” is intended to indicate that the mGluR5M polypeptide and the non-mGluR5M polypeptide are fused in-frame to each other. The non-mGluR5M polypeptide can be fused to the N-terminus or C-terminus of the mGluR5M polypeptide.

For example, in one embodiment, the fusion protein is a GST-mGluR5M fusion protein in which the mGluR5M sequences are fused to the C-terminus of the GST sequences. Such fusion proteins can facilitate the purification of recombinant mGluR5M. In another embodiment, the fusion protein is a mGluR5M protein containing a heterologous signal sequence at its N-terminus. In certain host cells (e.g., mammalian host cells), expression and/or secretion of mGluR5M can be increased through use of a heterologous signal sequence.

The mGluR5M fusion proteins of the invention can be incorporated into pharmaceutical compositions and administered to a subject in vivo. The mGluR5M fusion proteins can be used to affect the bioavailability of a mGluR5M substrate. Use of mGluR5M fusion proteins may be useful therapeutically for the treatment of central nervous system disorders. Moreover, the mGluR5M-fusion proteins of the invention can be used as immunogens to produce anti-mGluR5M antibodies in a subject, to purify mGluR5M ligands and in screening assays to identify molecules which inhibit the interaction of mGluR5M with a mGluR5M ligand or with a mGluR.

Preferably, a mGluR5M chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A mGluR5M-encoding nucleic acid can be cloned into such an expression vector such that the fusion moiety is linked in-frame to the mGluR5M protein.

The present invention also pertains to variants of the mGluR5M proteins which function as either mGluR5M agonists (mimetics) or as mGluR5M antagonists. Variants of the mGluR5M proteins can be generated by mutagenesis, e.g., discrete point mutation or truncation of a mGluR5M protein. An agonist of the mGluR5M proteins can retain substantially the same, or a subset, of the biological activities of the naturally occurring form of a mGluR5M protein. An antagonist of a mGluR5M protein can inhibit one or more of the activities of the naturally occurring form of the mGluR5M protein by, for example, competitively inhibiting the protease activity of a mGluR5M protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the mGluR5M protein.

In one embodiment, variants of a mGluR5M protein which function as either mGluR5M agonists (mimetics) or as mGluR5M antagonists can be identified by screening combinatorial libraries of mutants, e.g., truncation mutants, of a mGluR5M protein for mGluR5M protein agonist or antagonist activity. In one embodiment, a variegated library of mGluR5M variants is generated by combinatorial mutagenesis at the nucleic acid level and is encoded by a variegated gene library. A variegated library of mGluR5M variants can be produced by, for example, enzymatically ligating a mixture of synthetic oligonucleotides into gene sequences such that a degenerate set of potential mGluR5M sequences is expressible as individual polypeptides, or alternatively, as a set of larger fusion proteins (e.g., for phage display) containing the set of mGluR5M sequences therein. There are a variety of methods which can be used to produce libraries of potential mGluR5M variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential mGluR5M sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura etal. (1984) Science 198:1056; Ike et al. (1983) Nucleic Acid Res. 11:477.

In addition, libraries of fragments of a mGluR5M protein coding sequence can be used to generate a variegated population of mGluR5M fragments for screening and subsequent selection of variants of a mGluR5M protein. In one embodiment, a library of coding sequence fragments can be generated by treating a double stranded PCR fragment of a mGluR5M coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the mGluR5M protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of mGluR5M proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recrusive ensemble mutagenesis (REM), a new technique which enhances the frequency of functional mutants in the libraries, can be used in combination with the screening assays to identify mGluR5M variants (Arkin and Yourvan (1992) PNAS 89:7811-7815; Delgrave et al. (1993) Protein Engineering 6(3):327-331).

In one embodiment, cell based assays can be exploited to analyze a variegated mGluR5M library. For example, a library of expression vectors can be transfected into a cell line which ordinarily synthesizes mGluR5M. The transfected cells are then cultured such that a particular mutant mGluR5M is expressed and the effect of expression of the mutant on mGluR5M activity in the cell can be detected, e.g., by any of a number of activity assays for native mGluR5M protein. Plasmid DNA can then be recovered from the cells which score for modulated mGluR5M activity, and the individual clones further characterized.

An isolated mGluR5M protein, or a portion or fragment thereof, can be used as an immunogen to generate antibodies that bind mGluR5M using standard techniques for polyclonal and monoclonal antibody preparation. A full-length mGluR5M protein can be used or, alternatively, the invention provides antigenic peptide fragments of mGluR5M for use as immunogens. The antigenic peptide of mGluR5M comprises at least 8 amino acid residues of the amino acid sequence shown in SEQ ID NO:2 and encompasses an epitope of mGluR5M such that an antibody raised against the peptide forms a specific immune complex with mGluR5M. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of mGluR5M that are unique to the mGluR5M protein (e.g., within the C-terminal unique domain from about amino acids 304 to 369 of SEQ ID NO:2).

A mGluR5M immunogen typically is used to prepare antibodies by immunizing a suitable subject, (e.g., rabbit, goat, mouse or other mammal) with the immunogen. An appropriate immunogenic preparation can contain, for example, recombinantly expressed mGluR5M protein or a chemically synthesized mGluR5M polypeptide. The preparation can further include an adjuvant, such as Freund's complete or incomplete adjuvant, or similar immunostimulatory agent. Immunization of a suitable subject with an immunogenic mGluR5M preparation induces a polyclonal anti-mGluR5M antibody response.

Accordingly, another aspect of the invention pertains to anti-mGluR5M antibodies. The term “antibody” as used herein refers to immunoglobulin molecules and immunologically active portions of immunoglobulin molecules, i.e., molecules that contain an antigen binding site which specifically binds (immunoreacts with) an antigen, such as mGluR5M. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind mGluR5M. The term “monoclonal antibody” or “monoclonal antibody composition”, as used herein, refers to a population of antibody molecules that contain only one species of an antigen binding site capable of immunoreacting with a particular epitope of mGluR5M. A monoclonal antibody composition thus typically displays a single binding affinity for a particular mGluR5M protein with which it immunoreacts.

Polyclonal anti-mGluR5M antibodies can be prepared as described above by immunizing a suitable subject with a mGluR5M immunogen. The anti-mGluR5M antibody titer in the immunized subject can be monitored over time by standard techniques, such as with an enzyme linked immunosorbent assay (ELISA) using immobilized mGluR5M. If desired, the antibody molecules directed against mGluR5M can be isolated from the mammal (e.g., from the blood) and further purified by well known techniques, such as protein A chromatography to obtain the IgG fraction. At an appropriate time after immunization, e.g., when the anti-mGluR5M antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem 0.255:4980-83; Yeh et al. (1976) PNAS 76:2927-31; and Yeh et al. (1982) Int. J Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies. A New Dimension In Biological Analyses, Plenum Publishing Corp., New York, N.Y. (1980); E. A. Lemer (1981) Yale J Biol. Med., 54:387-402; M. L. Gefter et al (1977) Somatic Cell Genet. 3:231-36). Briefly, an immortal cell line (typically a myeloma) is fused to lymphocytes (typically splenocytes) from a mammal immunized with a mGluR5M immunogen as described above, and the culture supernatants of the resulting hybridoma cells are screened to identify a hybridoma producing a monoclonal antibody that binds mGluR5M.

Any of the many well known protocols used for fusing lymphocytes and immortalized cell lines can be applied for the purpose of generating an anti-mGluR5M monoclonal antibody (see, e.g., G. Galfre et al. (1977) Nature 266:55052; Gefter et al. Somatic Cell Genet., cited supra; Lemer, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind mGluR5M, e.g., using a standard ELISA assay.

Alternative to preparing monoclonal antibody-secreting hybridomas, a monoclonal anti-mGluR5M antibody can be identified and isolated by screening a recombinant combinatorial immunoglobulin library (e.g., an antibody phage display library) with mGluR5M to thereby isolate immunoglobulin library members that bind mGluR5M. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) PNAS 89:3576-3580; Garrad et al. (1991) Bio/Technology 9:1373-1377; Hoogenboom et al. (1991) Nuc. Acid Res. 19:4133-4137; Barbas et al. (1991) PNAS 88:7978-7982; and McCafferty et al. Nature (1990) 348:552-554.

Additionally, recombinant anti-mGluR5M antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) PNAS 84:3439-3443; Liu et al. (1987) J. Immunol. 139:3521-3526; Sun et al. (1987) PNAS 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-mGluR5M antibody (e.g., monoclonal antibody) can be used to isolate mGluR5M by standard techniques, such as affinity chromatography or immunoprecipitation. An anti-mGluR5M antibody can facilitate the purification of natural mGluR5M from cells and of recombinantly produced mGluR5M expressed in host cells. Moreover, an anti-mGluR5M antibody can be used to detect mGluR5M protein (e.g., in a cellular lysate or cell supernatant) in order to evaluate the abundance and pattern of expression of the mGluR5M protein. Anti-mGluR5M antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, -galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³³ I, ³⁵S or ³H.

III. Recombinant Expression Vectors and Host Cells

Another aspect of the invention pertains to vectors, preferably expression vectors, containing a nucleic acid encoding a mGluR5M protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., mGluR5M proteins, mutant forms of mGluR5M proteins, fusion proteins, etc.).

The recombinant expression vectors of the invention can be designed for expression of mGluR5M proteins in prokaryotic or eukaryotic cells. For example, mGluR5M proteins can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Phannacia, Piscataway, N.J.) which fuse glutathione S-transferase (GST), maltose E binding protein, or protein A, respectively, to the target recombinant protein.

Purified fusion proteins can be utilized in mGluR5M activity assays, (e.g., direct assays or competitive assays described in detail below), or to generate antibodies specific for mGluR5M proteins, for example. In a preferred embodiment, a mGluR5M fusion protein expressed in a retroviral expression vector of the present invention can be utilized to infect bone marrow cells which are subsequently transplanted into irradiated recipients. The pathology of the subject recipient is then examined after sufficient time has passed (e.g six (6) weeks).

Examples of suitable inducible non-fusion E. coli expression vectors include pTrc (Amann et al., (1988) Gene 69:301-315) and pET 11 d (Studier etal., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11 d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the mGluR5M expression vector is a yeast expression vector. Examples of vectors for expression in yeast S. cerivisae include pYepSec1 (Baldari, et al., (1987) Embo J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, mGluR5M proteins can be expressed in insect cells using baculovirus expression vectors. Baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to mGluR5M mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a mGluR5M protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding a mGluR5M protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) a mGluR5M protein. Accordingly, the invention further provides methods for producing a mGluR5M protein using the host cells of the invention. In one embodiment, the method comprises culturing the host cell of invention (into which a recombinant expression vector encoding a mGluR5M protein has been introduced) in a suitable medium such that a mGluR5M protein is produced. In another embodiment, the method further comprises isolating a mGluR5M protein from the medium or the host cell.

The host cells of the invention can also be used to produce nonhuman transgenic animals. For example, in one embodiment, a host cell of the invention is a fertilized oocyte or an embryonic stem cell into which mGluR5M-coding sequences have been introduced. Such host cells can then be used to create non-human transgenic animals in which exogenous mGluR5M sequences have been introduced into their genome or homologous recombinant animals in which endogenous mGluR5M sequences have been altered. Such animals are useful for studying the function and/or activity of a mGluR5M and for identifying and/or evaluating modulators of mGluR5M activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous mGluR5M gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a mGluR5M-encoding nucleic acid into the male pronuclei of a fertilized oocyte, e.g., by microinjection, retroviral infection, and allowing the oocyte to develop in a pseudopregnant female foster animal. The mGluR5M cDNA sequence, e.g., that of SEQ ID NO:1 or the nucleotide sequence of the DNA insert of the plasmid deposited with ATCC as Accession Number PTA-2775, can be introduced as a transgene into the genome of a non-human animal. Alternatively, a nonhuman orthologue of a human mGluR5M gene, such as a mouse or rat mGluR5M gene, can be used as a transgene. Alternatively, a mGluR5M gene homologue can be isolated based on hybridization to the mGluR5M cDNA sequences of SEQ ID NO:1 or the nucleotide sequence of the DNA insert of the plasmid deposited with ATCC as Accession Number PTA-2775 (described further in subsection I above) and used as a transgene. Intronic sequences and polyadenylation signals can also be included in the transgene to increase the efficiency of expression of the transgene. A tissue-specific regulatory sequence(s) can be operably linked to a mGluR5M transgene to direct expression of a mGluR5M protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a mGluR5M transgene in its genome and/or expression of mGluR5M mRNA in tissues or cells of the animals. A transgenic founder animal can then be used to breed additional animals carrying the transgene. Moreover, transgenic animals carrying a transgene encoding a mGluR5M protein can further be bred to other transgenic animals carrying other transgenes.

To create a homologous recombinant animal, a vector is prepared which contains at least a portion of a mGluR5M gene into which a deletion, addition or substitution has been introduced to thereby alter, e.g., functionally disrupt, the mGluR5M gene. The mGluR5M gene can be a human gene (e.g., the cDNA of SEQ ID NO:1), but more preferably, is a non-human homologue of a human mGluR5M gene (e.g., a cDNA isolated by stringent hybridization with the nucleotide sequence of SEQ ID NO:1). For example, a mouse mGluR5M gene can be used to construct a homologous recombination vector suitable for altering an endogenous mGluR5M gene in the mouse genome. In a preferred embodiment, the vector is designed such that, upon homologous recombination, the endogenous mGluR5M gene is functionally disrupted (i.e., no longer encodes a functional protein; also referred to as a “knock out” vector). Alternatively, the vector can be designed such that, upon homologous recombination, the endogenous mGluR5M gene is mutated or otherwise altered but still encodes functional protein (e.g., the upstream regulatory region can be altered to thereby alter the expression of the endogenous mGluR5M protein). In the homologous recombination vector, the altered portion of the mGluR5M gene is flanked at its 5′ and 3′ ends by additional nucleic acid sequence of the mGluR5M gene to allow for homologous recombination to occur between the exogenous mGluR5M gene carried by the vector and an endogenous mGluR5M gene in an embryonic stem cell. The additional flanking mGluR5M nucleic acid sequence is of sufficient length for successful homologous recombination with the endogenous gene. Typically, several kilobases of flanking DNA (both at the 5′ and 3′ ends) are included in the vector (see e.g., Thomas, K. R. and Capecchi, M. R. (1987) Cell 51:503 for a description of homologous recombination vectors). The vector is introduced into an embryonic stem cell line (e.g., by electroporation) and cells in which the introduced mGluR5M gene has homologously recombined with the endogenous mGluR5M gene are selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-humans animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) PNAS 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813. In brief, a cell, e.g., a somatic cell, from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The recontructed oocyte is then cultured such that it develops to morula or blastocyte and then transferred to pseudopregnant female foster animal. The offspring borne of this female foster animal will be a clone of the animal from which the cell, e.g., the somatic cell, is isolated.

IV. Pharmaceutical Compositions

The mGluR5M nucleic acid molecules, mGluR5M proteins, anti-mGluR5M antibodies, mGluR5M ligands, peptide, peptidomimetics and/or mGluR5M modulators (also referred to herein as “active compounds”) of the invention can be incorporated into pharmaceutical compositions suitable for administration. Such compositions typically comprise the nucleic acid molecule, protein, antibody, or modulatory compound and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a mGluR5M protein or anti-mGluR5M antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologues, peptides, antibodies and the like described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic). As described herein, a mGluR5M protein of the invention has one or more of the following activities: (1) modulation of G protein linked second messenger signaling pathways (e.g., modulation of diacylglycerol and/or inisitol triphosphate-mediated signaling pathways); (2) modulation of glutamatergic transmission; (3) modulation of neuronal excitability; (4) regulation of synaptic transmission; (5) modulation of neurotransmitter release (e.g., glutamate release); (6) regulation of voltage-dependent and/or voltage-independent and/or ligand-gated ion channels (e.g., K+channels or Ca2+ channels); (7) regulation of neuronal development (e.g., regulation of neuronal differentiation, migration and/or survival in the developing and/or mature brain); (8) modulation of neurodegenerative processes (e.g., acute or chronic neurodegenerative processes); and (9) modulation of mGluR5 dimerization (e.g., mGluR5a and/or mGluR5b dimerization) and/or modulation of dimerization of other mGluR family members (e.g., mGluR1 dimerization). Accordingly, the isolated nucleic acid molecules of the invention can be used, for example, to express mGluR5M protein (e.g., via a recombinant expression vector in a host cell in gene therapy applications), to detect mGluR5M mRNA (e.g., in a biological sample) or a genetic alteration in a mGluR5M gene, and to modulate mGluR5M activity, as described further below. The mGluR5M proteins can be used to treat disorders characterized by insufficient or excessive production of a mGluR5M protein and/or mGluR5M ligand. In addition, the mGluR5M proteins can be used to screen drugs or compounds which modulate the mGluR5M activity as well as to treat disorders characterized by insufficient or excessive production of mGluR5M protein or production of mGluR5M protein forms which have decreased or aberrant activity compared to mGluR5M wild type protein. Moreover, the anti-mGluR5M antibodies of the invention can be used to detect and isolate mGluR5M proteins, regulate the bioavailability of mGluR5M proteins, and modulate mGluR5M activity.

A. Screening Assays:

The invention provides a method (also referred to herein as a “screening assay”) for identifying modulators, i.e., candidate or test compounds or agents (e.g., peptides, peptidomimetics, small molecules or other drugs) which bind to mGluR5M proteins, or have a stimulatory or inhibitory effect on, for example, mGluR5M expression or mGluR5M activity.

In one embodiment, the invention provides assays for screening candidate or test compounds which bind to or modulate the activity of a mGluR5M protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho etal. (1993) Science 261:1303; Carrell etal. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner USP 5,223,409), spores (Ladner USP '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses a mGluR protein on the cell surface is contacted with a mGluR5M protein and a test compound and the ability of the test compound to modulate the binding of the mGluR5M protein to the mGluR determined. The cell, for example, can be of mammalian origin or a yeast cell. Determining the ability of the test compound to modulate the binding of the mGluR5M protein to the mGluR can be accomplished, for example, by coupling the test compound or the mGluR5M protein with a radioisotope or enzymatic label such that binding of the test compound or the mGluR5M protein to the mGluR protein can be determined by detecting the labeled compound in a complex. For example, test compounds can be labeled with ¹²⁵I, ³⁵S, ¹⁴C, or ³H, either directly or indirectly, and the radioisotope detected by direct counting of radioemmission or by scintillation counting. Alternatively, compounds or proteins can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a test compound to modulate interaction of a mGluR5M protein without the labeling of any of the interactants. For example, a microphysiometer can be used to detect the interaction of a test compound with a mGluR5M protein without the labeling of either the test compound or the mGluR5M protein. McConnell, H. M. et al. (1992) Science 257:1906-1912. As used herein, a “microphysiometer” (e.g., Cytosensor™) is an analytical instrument that measures the rate at which a cell acidifies its environment using a light-addressable potentiometric sensor (LAPS). Changes in this acidification rate can be used as an indicator of the interaction between ligand and receptor.

In a preferred embodiment, the assay comprises contacting a cell which expresses a mGluR5 protein on the cell surface with a mGluR5 ligand, to form an assay mixture, contacting the assay mixture with a mGluR5M protein or variant, optionally in addition to a test compound, and determining the ability of the test compound or the mGluR5M protein or variant to interact with the mGluR5 protein. In one embodiment, determining the ability of the mGluR5M protein or variant or test compound to interact with the mGluR5 protein comprises determining the ability of the mGluR5M protein or variant or test compound to preferentially bind to the mGluR5 protein, as compared to the ability of the mGluR5M to bind to the mGluR5 protein.

Determining the ability of the mGluR5M protein ortest compound to bind to or interact with a mGluR5 protein can be accomplished by one of the methods described above for determining direct binding. In a preferred embodiment, determining the ability of the mGluR5M protein or test compound to bind to or interact with a mGluR5 protein can be accomplished by determining the activity of the mGluR5 protein or of a downstream mGluR5 target molecule. For example, the target molecule can be a cellular second messenger, and the activity of the target molecule can be determined by detecting induction of of the target (i.e. intracellular Ca²⁺, diacylglycerol, IP₃, etc.), detecting catalytic/enzymatic activity of the target on an appropriate substrate, detecting the induction of a reporter gene (comprising a mGluR5-responsive regulatory element operatively linked to a nucleic acid encoding a detectable marker, e.g., luciferase), or detecting a cellular response, for example, a proliferative response or a neuronal response.

In yet another embodiment, an assay of the present invention is a cell-free assay in which a mGluR5M protein or biologically active portion thereof is contacted with a test compound and the ability of the test compound to bind to the mGluR5M protein or biologically active portion thereof is determined. Binding of the test compound to the mGluR5M protein can be determined either directly or indirectly as described above. Binding of the test compound to the mGluR5M protein can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore™). Changes in the optical phenomenon of surface plasmon resonance (SPR) can be used as an indication of real-time reactions between biological molecules.

In a preferred embodiment, the assay includes contacting the mGluR5M protein or biologically active portion thereof with a known ligand which binds mGluR5M to form an assay mixture, contacting the assay mixture with a test compound, and determining the ability of the test compound to interact with a mGluR5M protein, wherein determining the ability of the test compound to interact with a mGluR5M protein comprises determining the ability of the test compound to preferentially bind to mGluR5M or biologically active portion thereof as compared to the known ligand.

In another embodiment, the assay is a cell-free assay in which a mGluR5M protein or biologically active portion thereof is contacted with a test compound and the ability of the test compound to modulate (e.g., stimulate or inhibit) the activity of the mGluR5M protein or biologically active portion thereof is determined. Determining the ability of the test compound to modulate the activity of a mGluR5M protein can be accomplished, for example, by determining the ability of the mGluR5M protein to modulate the activity of a downstream mGluR5M target molecule by one of the methods described above for cell-based assays. For example, the catalytic/enzymatic activity of the target molecule on an appropriate substrate can be determined as previously described. Alternatively, the ability of mGluR5M to bind to or interact with a mGluR can be determined as previously described.

In yet another embodiment, the cell-free assay involves contacting a mGluR5M protein or biologically active portion thereof with a known ligand which binds the mGluR5M protein and optionally a mGluR protein, to form an assay mixture, contacting the assay mixture with a test compound, and determining the ability of the test compound to interact with the mGluR5M protein, wherein determining the ability of the test compound to interact with the mGluR5M protein comprises determining the ability of the test compound to preferentially bind to or modulate the activity of a mGluR5M target molecule, as compared to the known ligand.

The cell-free assays of the present invention are amenable to use of both soluble and/or membrane-bound forms of isolated proteins (e.g. mGluR5M proteins or mGluR5 proteins). In the case of cell-free assays in which a membrane-bound form an isolated protein is used (e.g., a mGluR5 protein) it may be desirable to utilize a solubilizing agent such that the membrane-bound form of the isolated protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In more than one embodiment of the above assay methods of the present invention, it may be desirable to immobilize either mGluR5M or its target molecule to facilitate separation of complexed from uncomplexed forms of one or both of the proteins, as well as to accommodate automation of the assay. Binding of a test compound to a mGluR5M protein, or interaction of a mGluR5M protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/mGluR5M fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or mGluR5M protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of mGluR5M binding or activity determined using standard techniques.

Other techniques for immobilizing proteins on matrices can also be used in the screening assays of the invention. For example, either a mGluR5M protein or a mGluR5M target molecule can be immobilized utilizing conjugation of biotin and streptavidin. Biotinylated mGluR5M protein or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with mGluR5M protein or target molecules but which do not interfere with binding of the mGluR5M protein to its target molecule can be derivatized to the wells of the plate, and unbound target or mGluR5M protein trapped in the wells by antibody conjugation. Methods for detecting such complexes, in addition to those described above for the GST-immobilized complexes, include immunodetection of complexes using antibodies reactive with the mGluR5M protein or target molecule, as well as enzyme-linked assays which rely on detecting an enzymatic activity associated with the mGluR5M protein or target molecule.

In another embodiment, modulators of mGluR5M expression are identified in a method wherein a cell is contacted with a candidate compound and the expression of mGluR5M mRNA or protein in the cell is determined. The level of expression of mGluR5M mRNA or protein in the presence of the candidate compound is compared to the level of expression of mGluR5M mRNA or protein in the absence of the candidate compound. The candidate compound can then be identified as a modulator of mGluR5M expression based on this comparison. For example, when expression of mGluR5M mRNA or protein is greater (statistically significantly greater) in the presence of the candidate compound than in its absence, the candidate compound is identified as a stimulator of mGluR5M mRNA or protein expression. Alternatively, when expression of mGluR5M mRNA or protein is less (statistically significantly less) in the presence of the candidate compound than in its absence, the candidate compound is identified as an inhibitor of mGluR5M mRNA or protein expression. The level of mGluR5M mRNA or protein expression in the cells can be determined by methods described herein for detecting mGluR5M mRNA or protein.

In yet another aspect of the invention, the mGluR5M proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with mGluR5M (“mGluR5M-binding proteins” or “mGluR5M-bp”) and are involved in mGluR5M activity. Such mGluR5M-binding proteins are also likely to be involved in the propagation of signals by the mGluR5M proteins as, for example, downstream elements of a mGluR5M-mediated signaling pathway. Alternatively, such mGluR5M-binding proteins are likely to be cell-surface molecules associated with non-mGluR5M expressing cells, wherein such mGluR5M-binding proteins are involved in chemoattraction.

The two-hybrid system is based on the modular nature of most transcription factors, which consist of separable DNA-binding and activation domains. Briefly, the assay utilizes two different DNA constructs. In one construct, the gene that codes for a mGluR5M protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a mGluR5M-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the mGluR5M protein.

This invention further pertains to novel agents identified by the above-described screening assays. Accordingly, it is within the scope of this invention to further use an agent identified as described herein in an appropriate animal model. For example, an agent identified as described herein (e.g., a mGluR5M modulating agent, an antisense mGluR5M nucleic acid molecule, a mGluR5M-specific antibody, or a mGluR5M-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. This process is called chromosome mapping. Accordingly, portions or fragments of the mGluR5M nucleotide sequences, described herein, can be used to map the location of the mGluR5M genes on a chromosome. The mapping of the mGluR5M sequences to chromosomes is an important first step in correlating these sequences with genes associated with disease.

Briefly, mGluR5M genes can be mapped to chromosomes by preparing PCR primers (preferably 15-25 bp in length) from the mGluR5M nucleotide sequences. Computer analysis of the mGluR5M sequences can be used to predict primers that do not span more than one exon in the genomic DNA, thus complicating the amplification process See e.g., Example 3, herein. These primers can then be used for PCR screening of somatic cell hybrids containing individual human chromosomes. Only those hybrids containing the human gene corresponding to the mGluR5M sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the mGluR5M nucleotide sequences to design oligonucleotide primers, sublocalization can be achieved with panels of fragments from specific chromosomes. Other mapping strategies which can similarly be used to map a 9o, 1p, or 1v sequence to its chromosome include in situ hybridization (described in Fan, Y. et al. (1990) PNAS, 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical such as colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the mGluR5M gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The mGluR5M sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the mGluR5M nucleotide sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The mGluR5M nucleotide sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:1, can comfortably provide positive individual identification with a panel of perhaps 10 to 1,000 primers which each yield a noncoding amplified sequence of 100 bases. If predicted coding sequences, such as those in SEQ ID NO:3 are used, a more appropriate number of primers for positive individual identification would be 500-2,000.

If a panel of reagents from mGluR5M nucleotide sequences described herein is used to generate a unique identification database for an individual, those same reagents can later be used to identify tissue from that individual. Using the unique identification database, positive identification of the individual, living or dead, can be made from extremely small tissue samples.

3. Use of Partial mGluR5M Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:1 are particularly appropriate for this use as greater numbers of polymorphisms occur in the noncoding regions, making it easier to differentiate individuals using this technique. Examples of polynucleotide reagents include the mGluR5M nucleotide sequences or portions thereof, e.g., fragments derived from the noncoding regions of SEQ ID NO:1, having a length of at least 20 bases, preferably at least 30 bases.

The mGluR5M nucleotide sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such mGluR5M probes can be used to identify tissue by species and/or by organ type.

In a similar fashion, these reagents, e.g., mGluR5M primers or probes can be used to screen tissue culture for contamination (i.e. screen for the presence of a mixture of different types of cells in a culture).

C. Predictive Medicine:

The present invention also pertains to the field of predictive medicine in which diagnostic assays, prognostic assays, and monitoring clinical trails are used for prognostic (predictive) purposes to thereby treat an individual prophylactically. Accordingly, one aspect of the present invention relates to diagnostic assays for determining mGluR5M protein and/or nucleic acid expression as well as mGluR5M activity, in the context of a biological sample (e.g., blood, serum, cells, tissue) to thereby determine whether an individual is afflicted with a disease or disorder, or is at risk of developing a disorder, associated with aberrant mGluR5M expression or activity. The invention also provides for prognostic (or predictive) assays for determining whether an individual is at risk of developing a disorder associated with mGluR5M protein, nucleic acid expression or activity. For example, mutations in a mGluR5M gene can be assayed in a biological sample. Such assays can be used for prognostic or predictive purpose to thereby phophylactically treat an individual prior to the onset of a disorder characterized by or associated with mGluR5M protein, nucleic acid expression or activity.

Another aspect of the invention pertains to monitoring the influence of agents (e.g., drugs, compounds) on the expression or activity of mGluR5M in clinical trials.

These and other agents are described in further detail in the following sections.

1. Diagnostic Assays

An exemplary method for detecting the presence or absence of mGluR5M protein or nucleic acid in a biological sample involves obtaining a biological sample from a test subject and contacting the biological sample with a compound or an agent capable of detecting mGluR5M protein or nucleic acid (e.g., mRNA, genomic DNA) that encodes mGluR5M protein such that the presence of mGluR5M protein or nucleic acid is detected in the biological sample. A preferred agent for detecting mGluR5M mRNA or genomic DNA is a labeled nucleic acid probe capable of hybridizing to mGluR5M mRNA or genomic DNA. The nucleic acid probe can be, for example, a full-length mGluR5M nucleic acid, such as the nucleic acid of SEQ ID NO:1, or a fragment or portion of a mGluR5M nucleic acid such as an oligonucleotide of at least 15, 30, 50, 100, 250 or 500 nucleotides in length and sufficient to specifically hybridize under stringent conditions to mGluR5M mRNA or genomic DNA. Other suitable probes for use in the diagnostic assays of the invention are described herein.

A preferred agent for detecting mGluR5M protein is an antibody capable of binding to mGluR5M protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect mGluR5M mRNA, protein, or genomic DNA in a biological sample in vitro as well as in vivo. For example, in vitro techniques for detection of mGluR5M mRNA include Northern hybridizations and in situ hybridizations. In vitro techniques for detection of mGluR5M protein include enzyme linked immunosorbent assays (ELISAs), Western blots, immunoprecipitations and immunofluorescence. In vitro techniques for detection of mGluR5M genomic DNA include Southern hybridizations. Furthermore, in vivo techniques for detection of mGluR5M protein include introducing into a subject a labeled anti-mGluR5M antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting mGluR5M protein, mRNA, or genomic DNA, such that the presence of mGluR5M protein, mRNA or genomic DNA is detected in the biological sample, and comparing the presence of mGluR5M protein, mRNA or genomic DNA in the control sample with the presence of mGluR5M protein, mRNA or genomic DNA in the test sample.

The invention also encompasses kits for detecting the presence of mGluR5M in a biological sample. For example, the kit can comprise a labeled compound or agent capable of detecting mGluR5M protein or mRNA in a biological sample; means for determining the amount of mGluR5M in the sample; and means for comparing the amount of mGluR5M in the sample with a standard. The compound or agent can be packaged in a suitable container. The kit can further comprise instructions for using the kit to detect mGluR5M protein or nucleic acid.

2. Prognostic Assays

The diagnostic methods described herein can furthermore be utilized to identify subjects having or at risk of developing a disease or disorder associated with aberrant mGluR5M or mGluR expression or activity. For example, the assays described herein, such as the preceding diagnostic assays or the following assays, can be utilized to identify a subject having or at risk of developing a disorder associated with mGluR5M protein, nucleic acid expression or activity such as a CNS or psychiatric disorder. Alternatively, the prognostic assays can be utilized to identify a subject having or at risk for developing a CNS or psychiatric disorder. Thus, the present invention provides a method for identifying a disease or disorder associated with aberrant mGluR5M or mGluR expression or activity in which a test sample is obtained from a subject and mGluR5M protein or nucleic acid (e.g, mRNA, genomic DNA) is detected, wherein the presence of mGluR5M protein or nucleic acid is diagnostic for a subject having or at risk of developing a disease or disorder associated with aberrant mGluR5M or mGluR expression or activity. As used herein, a “test sample” refers to a biological sample obtained from a subject of interest. For example, a test sample can be a biological fluid (e.g., serum), cell sample, or tissue, in particular a neuronal cell sample or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant mGluR5M expression or activity. For example, such methods can be used to determine whether a subject can be effectively treated with an agent for a disorder, such as a CNS or psychiatric disorder. Alternatively, such methods can be used to determine whether a subject can be effectively treated with an agent for an inflammatory disease. Thus, the present invention provides methods for determining whether a subject can be effectively treated with an agent for a disorder associated with aberrant mGluR5M or mGluR expression or activity in which a test sample is obtained and mGluR5M protein or nucleic acid expression or activity is detected (e.g., wherein the abundance of mGluR5M protein or nucleic acid expression or activity is diagnostic for a subject that can be administered the agent to treat a disorder associated with aberrant mGluR5M or mGluR expression or activity.)

The methods of the invention can also be used to detect genetic alterations in a mGluR5M gene, thereby determining if a subject with the altered gene is at risk for a disorder characterized by an aberrant inflammatory response. In preferred embodiments, the methods include detecting, in a sample of cells from the subject, the presence or absence of a genetic alteration characterized by at least one of an alteration affecting the integrity of a gene encoding a mGluR5M-protein, or the mis-expression of the mGluR5M gene. For example, such genetic alterations can be detected by ascertaining the existence of at least one of 1) a deletion of one or more nucleotides from a mGluR5M gene; 2) an addition of one or more nucleotides to a mGluR5M gene; 3) a substitution of one or more nucleotides of a mGluR5M gene, 4) a chromosomal rearrangement of a mGluR5M gene; 5) an alteration in the level of a messenger RNA transcript of a mGluR5M gene, 6) aberrant modification of a mGluR5M gene, such as of the methylation pattern of the genomic DNA, 7) the presence of a non-wild type splicing pattern of a messenger RNA transcript of a mGluR5M gene, 8) a non-wild type level of a mGluR5M-protein, 9) allelic loss of a mGluR5M gene, and 10) inappropriate post-translational modification of a mGluR5M-protein. As described herein, there are a large number of assay techniques known in the art which can be used for detecting alterations in a mGluR5M gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) PNAS 91:360-364), the latter of which can be particularly useful for detecting point mutations in the mGluR5M-gene (see Abravaya et al. (1995) Nucleic Acids Res 0.23:675-682). This method can include the steps of collecting a sample of cells from a patient, isolating nucleic acid (e.g., genomic, mRNA or both) from the cells of the sample, contacting the nucleic acid sample with one or more primers which specifically hybridize to a mGluR5M gene under conditions such that hybridization and amplification of the mGluR5M-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), Q-Beta Replicase (Lizardi, P. M. et all, 1988, Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a mGluR5M gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in mGluR5M can be identified by hybridizing a sample and control nucleic acids, e.g., DNA or RNA, to high density arrays containing hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in mGluR5M can be identified in two dimensional arrays containing light-generated DNA probes as described in Cronin, M. T. et al supra. Briefly, a first hybridization array of probes can be used to scan through long stretches of DNA in a sample and control to identify base changes between the sequences by making linear arrays of sequential ovelapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the mGluR5M gene and detect mutations by comparing the sequence of the sample mGluR5M with the corresponding wild-type (control) sequence. Examples of sequencing reactions include those based on techniques developed by Maxim and Gilbert ((1977) PNAS 74:560) or Sanger ((1977) PNAS 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen etal. (1996) Adv. Chromatogr. 36:127-162; and Griffin et al. (1993) Appl. Biochem. Biotechnol. 38:147-159).

Other methods for detecting mutations in the mGluR5M gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type mGluR5M sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc. Natl Acad Sci USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in mGluR5M cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a mGluR5M sequence, e.g., a wild-type mGluR5M sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in mGluR5M genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat Res 285:125-144; and Hayashi (1992) Genet Anal Tech Appl 9:73-79). Single-stranded DNA fragments of sample and control mGluR5M nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a mGluR5M gene.

Furthermore, any cell type or tissue in which mGluR5M is expressed may be utilized in the prognostic assays described herein.

3. Monitoring of Effects During Clinical Trials

Monitoring the influence of agents (e.g., drugs, compounds) on the expression or activity of a mGluR5M protein (e.g., modulation of a CNS or psychiatric responese) an be applied not only in basic drug screening, but also in clinical trials. For example, the effectiveness of an agent determined by a screening assay as described herein to increase mGluR5M gene expression, protein levels, or upregulate mGluR5M or mGluR activity, can be monitored in clinical trails of subjects exhibiting decreased mGluR5M gene expression, protein levels, or downregulated mGluR5M or mGluR activity. Alternatively, the effectiveness of an agent determined by a screening assay to decrease mGluR5M gene expression, protein levels, or downregulate mGluR5M or mGluR activity, can be monitored in clinical trails of subjects exhibiting increased mGluR5M gene expression, protein levels, or upregulated mGluR5M or mGluR activity. In such clinical trials, the expression or activity of a mGluR5M gene, and preferably, other genes that have been implicated in, for example, an inflammatory disorder can be used as a “read out” or markers of the phenotype of a particular cell.

For example, and not by way of limitation, genes, including mGluR5M, that are modulated in cells by treatment with an agent (e.g., compound, drug or small molecule) which modulates mGluR5M activity (e.g., identified in a screening assay as described herein) can be identified. Thus, to study the effect of agents on CNS or psychiatric disorders, for example, in a clinical trial, cells can be isolated and RNA prepared and analyzed for the levels of expression of mGluR5M and other genes implicated in the CNS or psychiatric disorder, respectively. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of mGluR5M or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of expression of a mGluR5M protein, mRNA, or genomic DNA in the preadministration sample; (iii) obtaining one or more post-administration samples from the subject; (iv) detecting the level of expression or activity of the mGluR5M or mGluR protein, mRNA, or genomic DNA in the post-administration samples; (v) comparing the level of expression or activity of the mGluR5M or mGluR protein, mRNA, or genomic DNA in the pre-administration sample with the mGluR5M protein, mRNA, or genomic DNA in the post administration sample or samples; and (vi) altering the administration of the agent to the subject accordingly. For example, increased administration of the agent may be desirable to increase the expression or activity of mGluR5M to higher levels than detected, i.e., to increase the effectiveness of the agent. Alternatively, decreased administration of the agent may be desirable to decrease expression or activity of mGluR5M to lower levels than detected, i.e. to decrease the effectiveness of the agent. According to such an embodiment, mGluR5M expression or activity may be used as an indicator of the effectiveness of an agent, even in the absence of an observable phenotypic response.

D. Methods of Treatment:

The present invention provides for both prophylactic and therapeutic methods of treating a subject at risk of (or susceptible to) a disorder or having a disorder associated with aberrant mGluR5M or mGluR expression or activity. With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with either the mGluR5M molecules of the present invention or mGluR5M modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

1. Prophylactic Methods

In one aspect, the invention provides a method for preventing in a subject, a disease or condition associated with an aberrant mGluR5M or mGluR expression or activity, by administering to the subject a mGluR5M or an agent which modulates mGluR5M expression or at least one mGluR5M or mGluR activity. Subjects at risk for a disease which is caused or contributed to by aberrant mGluR5M expression or activity can be identified by, for example, any or a combination of diagnostic or prognostic assays as described herein. Administration of a prophylactic agent can occur prior to the manifestation of symptoms characteristic of the mGluR5M aberrancy, such that a disease or disorder is prevented or, alternatively, delayed in its progression. Depending on the type of mGluR5M aberrancy, for example, a mGluR5M, mGluR5M agonist or mGluR5M antagonist agent can be used for treating the subject. The appropriate agent can be determined based on screening assays described herein. The prophylactic methods of the present invention are further discussed in the following subsections.

2. Therapeutic Methods

Another aspect of the invention pertains to methods of modulating mGluR5M expression or activity for therapeutic purposes. Accordingly, in an exemplary embodiment, the modulatory method of the invention involves contacting a cell with a mGluR5M molecule of the present invention such that the activity of a mGluR5M is modulated. Alternatively, the modulatory method of the invention involves contacting a cell with an agent that modulates one or more of the activities of mGluR5M protein activity associated with the cell. An agent that modulates mGluR5M protein activity can be an agent as described herein, such as a nucleic acid or a protein, a naturally-occurring target molecule of a mGluR5M protein, a mGluR5M antibody, a mGluR5M agonist or antagonist, a peptidomimetic of a mGluR5M agonist or antagonist, or other small molecule. In one embodiment, the agent stimulates one or more mGluR5M activites. Examples of such stimulatory agents include active mGluR5M protein and a nucleic acid molecule encoding mGluR5M that has been introduced into the cell. In another embodiment, the agent inhibits one or more mGluR5M activites. Examples of such inhibitory agents include antisense mGluR5M nucleic acid molecules and anti-mGluR5M antibodies. These modulatory methods can be performed in vitro (e.g., by culturing the cell with the agent) or, alternatively, in vivo (e.g, by administering the agent to a subject). As such, the present invention provides methods of treating an individual afflicted with a disease or disorder characterized by aberrant expression or activity of a mGluR5M protein or nucleic acid molecule. In one embodiment, the method involves administering an agent (e.g., an agent identified by a screening assay described herein), or combination of agents that modulates (e.g., upregulates or downregulates) mGluR5M expression or activity. In another embodiment, the method involves administering a mGluR5M protein or nucleic acid molecule as therapy to compensate for reduced or aberrant mGluR5M expression or activity.

Stimulation of mGluR5M activity is desirable in situations in which mGluR5M is abnormally downregulated and/or in which increased mGluR5M activity is likely to have a beneficial effect (e.g., in situations where mGluR activity is abnormally upregulated or increased, for example, in a CNS or psychiatric disorder). Likewise, inhibition of mGluR5M activity is desirable in situations in which mGluR5M is abnormally upregulated and/or in which decreased mGluR5M activity is likely to have a beneficial effect.

3. Pharmacogenomics

The mGluR5M molecules of the present invention, as well as agents, or modulators which have a stimulatory or inhibitory effect on mGluR5M activity (e.g., mGluR5M gene expression) as identified by a screening assay described herein can be administered to individuals to treat (prophylactically or therapeutically) disorders (e.g, in CNS or psychiatric disorders) associated with aberrant mGluR5M activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a mGluR5M molecule or mGluR5M modulator as well as tailoring the dosage and/or therapeutic regimen of treatment with a mGluR5M molecule or mGluR5M modulator.

Pharmacogenomics deals with clinically significant hereditary variations in the response to drugs due to altered drug disposition and abnormal action in affected persons. See e.g., Eichelbaum, M., Clin Exp Pharmacol Physiol, 1996, 23(10-11):983-985 and Linder, M. W., Clin Chem, 1997, 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., a mGluR5M protein or mGluR5M protein of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C 19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C 19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., a mGluR5M molecule or mGluR5M modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a mGluR5M molecule or mGluR5M modulator, such as a modulator identified by one of the exemplary screening assays described herein.

This invention is further illustrated by the following examples which should not be construed as limiting. The contents of all references, patents and published patent applications cited throughout this application are hereby incorporated by reference. References throughout the instant specification to websites maintained as part of the World Wide Web are referred to herein by the prefix http://. The information contained in such websites is publically-avialable and can be accessed elctronically by contacting the cited address.

### EXAMPLES

### Example 1

### Identification and Characterization of a mGluR5M cDNA

In this example, the identification and characterization of the genes encoding human mGluR5M (also referred to interchangeably herein as “YI176”) is described.

Isolation of the Human mGluR5M cDNAs

A fill-length clone designated YI176 was identified in a cDNA library derived from adult human brain mRNA (Clonetech™). (A partial YI176 cDNA was also isolated from a Clonetech™ hippocampus cDNA library.) The human clone contained an insert of approximately 1823 bp containing a protein-encoding sequence (i.e., an open reading frame) of approximately 1110 nucleotides capable of encoding approximately 369 amino acids of mGluR5M.

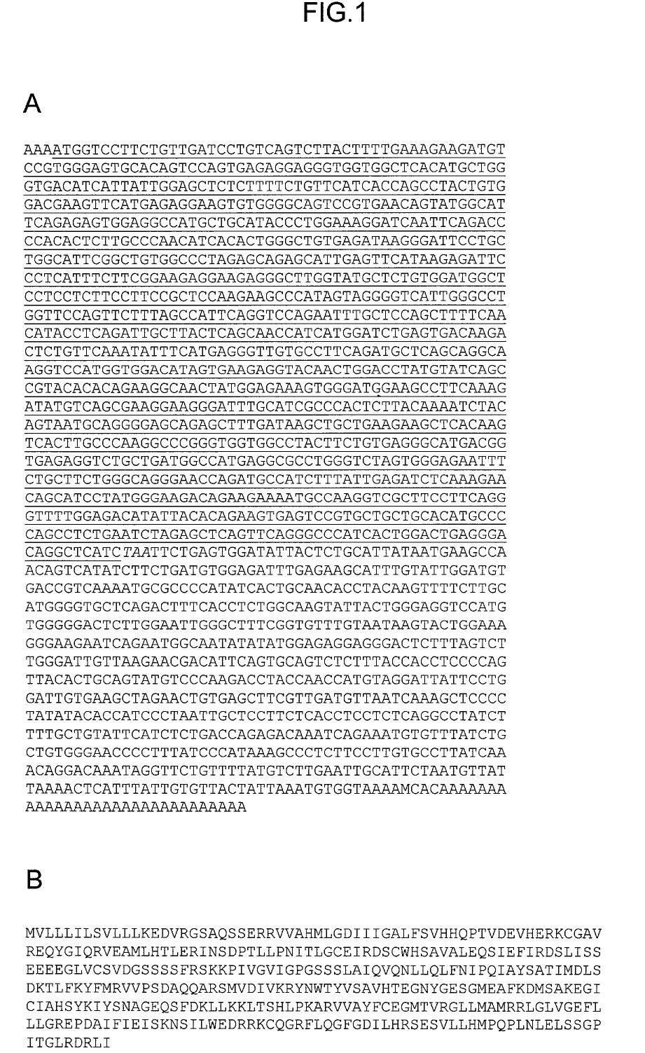

The nucleotide sequence encoding the human mGluR5M protein is shown in FIG. 1 and is set forth as SEQ ID NO:1. The full length protein encoded by this nucleic acid includes about 369 amino acids and has the amino acid sequence shown in FIG. 1 and set forth as SEQ ID NO:2. The coding portion (open reading frame) of SEQ ID NO:1 is set forth as SEQ ID NO:3.

Analysis of Human mGluR5M

A BLAST search (Altschul et al. (1990) J. Mol. Biol. 215:403) of the amino acid sequence of human mGluR5M revealed that mGluR5M is significantly similar to the N-terminus of the human metabotropic glutamate receptor 5 (mGluR5). In particular, the mGluR5M protein identified has a N-terminus which is nearly identical to the N-terminal extracellular binding domain of the mGluR5 protein (e.g., SwissProt™ Accession No. P41594). Amino acids 1-303 of mGluR5M are ˜97% identical to the N-terminal extracellular domain of mGluR5a and/or mGluR5b. By contrast, amino acids 304-369 are unique as determined by BLAST analysis.

A more recent BLAST search identified a cDNA termed RNF18 (a testis-specific ring finger protein having GenBank Accession No. AB037682 (Yoshikawa et al (2000) BBA 1493:349-355)) as sharing identity with YI176 at the 3′ end. Moreover, ESTs have been reported which contain sequence that spans the YI176/mGluR5 region (GBESTHUM3_REL:BE674422and GBESTHUM3_REL:BE467477), evidencing that YI176 represents a true mRNA (in contrast to a library artifact, for example, an mGluR5 artifact).

A Prosite search resulted in the identification of a G protein receptor family 3_{—}1 consensus sequence at about amino acids 158-176 of SEQ ID NO:2. mGluR5M is also predicted to contain Asn glycosylation sites at about amino acids 88-91 and 210-213 of SEQ ID NO:2.

Tissue Distribution of mGluR5M mRNA

This Example describes the tissue distribution of mGluR5M mRNA, as determined by Northern blot analysis.

Northern blot hybridizations with various RNA samples were performed as follows. Clonetech Human Multiple Tissue Northerns™ and Human Multiple Tissue Array™ membranes were probed using the manufacturer's protocols and ³²P-labeled DNA probes. Probes containing YI176-specific sequence (nucleotides 959-1103 and nucleotides 1481-1846 of SEQ ID NO:1) were generated as follows. Plasmid DNA from an isolated YI176 colony was prepared using the QIAprep Spin Miniprep™ Kit and manufacturer's protocol. Subsequently, the DNA was restriction digested with PstI and NotI or BgIII and ApaI according to the manufacturer's instructions. Restriction fragments were size-fractionated by gel electrophoresis on 1.5% agarose, 0.1 μg/ml ethidium bromide, 1× gels (Maniatis et al., 1982). The ethidium-bromide stained DNA band of the appropraite size (PstI/NotI˜365 bp; BgIII/ApaI˜144 bp) was excised from the agarose gel. Next, the DNA was extracted from the agarose using the Clonetech NucleoSpin™ Nucleic Acid Purification Kit and manufacturer's protocol. The extracted DNA was labeled with Redivue™ (α³²P)dCTP using the Prime-t II™ Random Primer Labeling Kit and protocol. Unincorporated (α³²P)dCTP was removed with Amersham's NICK™ column and protocol. Membranes were prehybridized to block non-specific binding interactions and subsequently hybridized with an appropriate aliquot of the ³²P-labeled YI176 probe under standard conditions. After hybridization and washing, membranes were air dried, exposed to X-ray film and subsequently developed and analyzed.

No bands were detected upon analysis of the Multiple Tissue Northerns™. However, the Multiple Tissue Array™ membranes indicated that a YI176 is expressed in neural but not heart or other non-neural tissues. These data in addition to those obtained from further Northern analysis indicate that mGluR5M mRNA is detectable in whole brain, cerebral cortex, frontal lobe, parietal lobe, occipital lobe, temporal lobe, paracentral gyrus of cerebral cortex, pons, cerebellum, corpus callosum, amygdala, caudate nucleus, hippocampus, medulla oblongta, putamen, substantia nigra, accumbens nucleus, thalamus and fetal brain. Notably, expression of mGluR5M is prdominant in cells and/or tissues of the central nervous system.

### Example 2

### Identification and Characterization of a mGluR5M cDNA

In this example, the chromosomal mapping of the gene encoding human mGluR5M (also referred to interchangeably herein as “YI176”) is described.

The chromosomal location of YI176 and mGluR5 was determined by BLAST™ searching of the HTGS databases using the appropriate relative query sequences. These analyses indicated that mGluR5 maps to BACs assigned to chromosome 11 and that YI176 maps to BACs assigned to chromosome 11 and chromosome 3. Of particular interest is the fact that at least some of the YI176-encoding BACs map to a region of chromosome 11 that has recently been identified as one of the regions affected in a balanced translocation event associated with schizophrenia and related psychiatric disorders (Millar et al. (2000) Hum. Mol. Genet. 9:1415-1423). In particular, Millar and co-workers described a balanced (1;11)(q42.1;q14.3) translocation which segregates with schizophrenia and related psychiatric disorders in a large Scottish family studied. Millar and co-workers analyzed the affected region on chromosome 1 and, in particular, described two novel genes, Disrupted-In-Schizophrenia 1 and 2 (DISC1 and DISC2), believed to play a role in nervous system function and/or susceptibility to psychiatric illness. Millar and co-workers described the affected chromosome 11 region as being characterized by a dearth of genes in the breakpoint region, concluding that it is unlikely that the expression of any genes on this chromosome are affected by the translocation. The above-described chromosimal mapping data (e.g., radiation hybrid mapping), the specific expression patterns of YI176 mRNA, as well as genomic predictions indicate the gene as a candidate for schizophrenia and/or other psychiatric disorders.

### Example 3

### Radiation Hybrid Mapping of YI176

Radiation Hybrid mapping was performed using the GeneBridge 4 Radiation Hybrid Panel (catalog # RH02.02 Research Genetics, Inc) and manufacturer's protocol. Briefly, polymerase chain reaction (PCR) amplification with the primers 5′TGCTGCTGCACATGCCCC and 5′TTAGATGAGCCTGTCCCTCAGTCC was performed using template DNA from each of the somatic hybrid clones. A reaction mixture was compiled with components at the following final concentrations: 50ng of DNA, 8 pmol of each; 0.2 mM each dATP, dTTP, dCTP, and dGTP (Amersham Pharmacia); one unit AmpliTaqGold™ polymerase; 1× reaction buffer (Applied Biosystems); 2.5 mM MgCl2. The mixture was incubated at 95° C. for ten minutes, followed by 30 cycles of 94° C. 30 seconds, 65° C. 30 seconds, 72° C. 23 seconds (MJResearch DNA Engine Tetrad PTC-225). PCR amplification products were size-fractionated on 3% agarose, 1× TAE gels (Maniatis et al). Each somatic hybrid clone was scored for the presence or absence of the PCR product. The results were submitted to http://www-genome.wi.mit.edu/cgi-bin/contig/rhmapper.p1 or http://www.sanger.ac.uk/Software/RHserver/RHserver.shtml for RH mapping. The MIT mapping server assigned the gene to chromosome 11, 31.33cR from the marker D11S1350. The Sanger mapping server assigned the gene to chromsome 11 between the markers AFMa131xd5 and AFM344zg1.

### Example 4

### Expression of Recombinant YI176 Protein in HEK293 Cells

YI176 cDNA was stably transfected into HEK293 cells using the Invitrogen™ FLP-IN system according to manufacturer's instructions. YI176 protein (epitope tagged for detection) was detectable in the medium confirming that the protein is secreted, as was predicted from sequence analysis.

### Example 5

### Heterodimer Formation of Recombinant YI176 Protein with mGluR5

Transient co-transfections into HEK293 cells were performed using the Lipofectamine Plus™ system (InVitrogen) according to manufacturer's instructions. Sample 1: Cells co-transfected with a cDNA encoding a V5-tagged secreted form of mGluR5 (comprising mGluR5 residues 1-576 which includes the extracellular domain but lacks the transmembrane domains), referred to herein as rtV5, and full-length (FL) mGluR5. Sample 2: Cells transfected with rtV5 alone. Sample 3: Cells transfected with YI176-V5 (tagged YI176) alone. Sample 4: Cells co-transfected with YI176-V5 and FL mGluR5. Sample 5: Cells transfected with FL mGluR5 alone. Sample 6: Mock-transfected cells.

Cell membrane fractions were prepared from the transfected cells in accordance will well-established protocols, i.e., cell lysis followed by washing and centrifugation steps to separate cytosolic, nuclear, and membrane fractions. Proteins were immunoprecipitated from cell membrane fractions with an anti-mGluR5 antibody, then separated by SDS polyacrylamide gel electrophoresis. Western blot analysis was performed using an anti-V5 tag-specific antibody. The results are set forth in Table 1.

TABLE 1
IP with anti-m GluR5 antibody; Western blot with anti-V5 antibody

| | rtV5 + | | | YI176-V5 + | | |
|---|---|---|---|---|---|---|
| | FL mGluR5 | rtV5 | YI176-V5 | FL mGluR5 | FL mGluR5 | MOCK |
| V5 tagged | +++++ | | | | | |
| mGluR5 | | | | | | |
| YI176-V5 | | | | ++++ | | |

V5-tagged (secreted) mGluR5:FL mGluR5 complexes were detected in Sample 1, indicating dimerization of the tagged (secreted) mGluR5 with FL mGluR5 receptor expressed in cell membranes. Notably, YI176:FL mGluR5 complexes were similarly detected in Sample 4, demonstrating that YI176 is able to dimerize with FL mGluR5 expressed in cell membranes.

Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. An isolated nucleic acid molecule comprising a nucleotide sequence at least 80% identical to the nucleotide sequence of SEQ ID NO:3, wherein the nucleic acid molecule encodes polypeptide comprising an N-terminal mGluR-like domain and a C-terminal unique domain.

2. The nucleic acid molecule of claim 1, comprising a nucleotide sequence at least 90% identical to the nucleotide sequence of SEQ ID NO:3.

3. The nucleic acid molecule of claim 1, comprising a nucleotide sequence at least 95% identical to the nucleotide sequence of SEQ ID NO:3.

4. An isolated nucleic acid molecule which encodes a polypeptide comprising an amino acid sequence at least 80% identical to the amino acid sequence of SEQ ID NO:2, wherein the polypeptide comprises an N-terminal mGluR-like domain and a C-terminal unique domain.

5. An isolated nucleic acid molecule which encodes a polypeptide comprising an amino acid sequence at least 80% identical to the amino acid sequence of SEQ ID NO:2, wherein the polypeptide lacks a transmembrane domain.

6. An isolated nucleic acid molecule which encodes a polypeptide comprising an amino acid sequence at least 80% identical to the amino acid sequence of SEQ ID NO:2, wherein percent identity is determined using a global alignment algorithm.

7. The nucleic acid molecule of claim 6, wherein percent identity is determined according to the ALIGN algorithm using a PAM120 weight residue table, a gap length penalty of 12 and a gap penalty of 4.

8. The nucleic acid molecule of any one of claims 4-7, wherein the amino acid sequence encoded is at least 90% identical to the amino acid sequence of SEQ ID NO:2.

9. The nucleic acid molecule of any one of claims 4-7, wherein the amino acid sequence encoded is at least 95% identical to the amino acid sequence of SEQ ID NO:2.

10. An isolated nucleic acid molecule which hybridizes to a complement of a nucleic acid molecule comprising SEQ ID NO:3 under stringent conditions, wherein the nucleic acid molecule encodes polypeptide comprising an N-terminal mGluR-like domain and a C-terminal unique domain.

11. An isolated nucleic acid molecule which hybridizes to a complement of a nucleic acid molecule comprising SEQ ID NO:1 under stringent conditions, wherein the nucleic acid molecule encodes a polypeptide lacking a transmembrane domain.

12. An isolated nucleic acid molecule comprising the DNA insert of the plasmid deposited with ATCC as Accession Number PTA-2775.

13. An isolated nucleic acid molecule comprising the nucleotide sequence of SEQ ID NO:1, or a complement thereof.

14. An isolated nucleic acid molecule which encodes a polypeptide comprising the amino acid sequence of SEQ ID NO:2.

15. The nucleic acid molecule of any one of claims 1, 4-6 and 10-14, further comprising vector nucleic acid sequences.

16. The nucleic acid molecule of any one of claims 1, 4-6 and 10-14, further comprising nucleic acid sequences encoding a heterologous polypeptide.

17. A host cell which contains the nucleic acid molecule of any one of claims 1, 4-6 and 10-14.

18. The host cell of claim 17 which is a mammalian host cell.

19. A non-human mammalian host cell comprising the nucleic acid molecule of claim any one of claims 1, 4-6 and 10-14.

20. An isolated polypeptide encoded by a nucleic acid molecule comprising a nucleotide sequence at least 80% identical to the nucleotide sequence of SEQ ID NO:3, wherein the polypeptide comprises an N-terminal mGluR-like domain and a C-terminal unique domain.

21. The polypeptide of claim 20, encoded by a nucleic acid molecule comprising a nucleotide sequence at least 90% identical to the nucleotide sequence of SEQ ID NO:3.

22. The polypeptide of claim 20, encoded by a nucleic acid molecule comprising a nucleotide sequence at least 95% identical to the nucleotide sequence of SEQ ID NO:3.

23. An isolated polypeptide comprising an amino acid sequence at least 80% identical to the amino acid sequence of SEQ ID NO:2, wherein the polypeptide comprises an N-terminal mGluR-like domain and a C-terminal unique domain.

24. An isolated polypeptide comprising an amino acid sequence at least 80% identical to the amino acid sequence of SEQ ID NO:2, wherein the polypeptide lacks a transmembrane domain.

25. An isolated polypeptide comprising an amino acid sequence at least 80% identical to the amino acid sequence of SEQ ID NO:2, wherein percent identity is determined using a global alignment algorithm.

26. The polypeptide of claim 25, wherein percent identity is determined according to the ALIGN algorithm using a PAM120 weight residue table, a gap length penalty of 12 and a gap penalty of 4.

27. The polypeptide of any one of claims 23-26, comprising an amino acid sequence at least 90% identical to the amino acid sequence of SEQ ID NO:2.

28. The polypeptide of any one of claims 23-26, comprising an amino acid sequence at least 95% identical to the amino acid sequence of SEQ ID NO:2.

29. An isolated polypeptide encoded by a nucleic acid molecule which hybridizes to a complement of a nucleic acid molecule comprising SEQ ID NO:1 under stringent conditions, wherein polypeptide comprises an N-terminal mGluR-like domain and a C-terminal unique domain.

30. An isolated polypeptide encoded by a nucleic acid molecule which hybridizes to a complement of a nucleic acid molecule comprising SEQ ID NO:1 under stringent conditions, wherein the nucleic acid molecule encodes a polypeptide lacking a transmembrane domain.

31. An isolated polypeptide encoded by the DNA insert of the plasmid deposited with ATCC as Accession Number PTA-2775.

32. An isolated polypeptide encoded by a nucleic acid molecule comprising the nucleotide sequence of SEQ ID NO:1.

33. An polypeptide comprising the amino acid sequence of SEQ ID NO:2.

34. The polypeptide of claim any one of claims 20, 23-25 and 29-33, further comprising heterologous amino acid sequences.

35. An antibody which selectively binds to the polypeptide of any one of claims 20, 23-25 and 29-33.

36. A method for producing a polypeptide encoded by the nucleic acid molecule of any one of claims 1, 4-6 and 10-14, comprising culturing a host cell which contains the nucleic acid molecule, under conditions in which the nucleic acid molecule is expressed.

37. A method for detecting the presence of the polypeptide of claim any one of claims 20, 23-25 and 29-33 in a sample comprising:
a) contacting the sample with a compound which selectively binds to the polypeptide; and
b) determining whether the compound binds to the polypeptide in the sample to thereby detect the presence of the polypeptide in the sample.

38. The method of claim 37, wherein the compound which binds to the polypeptide is an antibody.

39. A kit comprising a compound which selectively binds to a polypeptide of any one of claims 20, 23-25 and 29-33 and instructions for use.

40. A method for detecting the presence of the nucleic acid molecule of any one of claims 1, 4-6 and 10-14 in a sample comprising:
a) contacting the sample with a nucleic acid probe or primer which selectively hybridizes to the nucleic acid molecule; and
b) determining whether the nucleic acid probe or primer binds to a nucleic acid molecule in the sample to thereby detect the presence of the nucleic acid molecule in the sample.

41. The method of claim 40, wherein the sample comprises mRNA molecules and is contacted with a nucleic acid probe.

42. A kit comprising a compound which selectively hybridizes to the nucleic acid molecule of any one of claims 1, 4-6 and 10-14 and instructions for use.

43. A method for identifying a compound which modulates mGluR activity in a cell comprising:
a) contacting a cell expressing the mGluR with the polypeptide of any one of claims 20, 23-25 and 29-33 and a test compound; and
b) determining whether the test compound modulates the ability of the polypeptide to modulate mGluR activity as compared to an appropriate control.

44. A method for modulating the activity of a mGluR comprising contacting a cell expressing the mGluR with the polypeptide of any one of claims 20, 23-25 and 29-33, or a modulator of said polypeptide, in a sufficient concentration to modulate the activity of the mGluR.

45. A method for modulating neuronal cell signaling comprising contacting a neuronal cell with the polypeptide of any one of claims 20, 23-25 and 29-33, or a modulator of said polypeptide, in a sufficient concentration to modulate at least one signaling pathway of the neuronal cell.

46. The method of claim 45, wherein the neuronal cell expresses an mGluR receptor.

47. The method of claim 46, wherein the mGluR5 receptor is mGluR5a or mGlu5b.

48. A method for treating a subject having a neurological disorder comprising administering to said subject an effective amount of the polypeptide of claim any one of claims 20, 23-25 and 29-33, or a modulator of said polypeptide, such that the neurological disorder is treated.

49. A method for treating a subject having a psychiatric disorder comprising administering to said subject an effective amount of the polypeptide of any one of claims 20, 23-25 and 29-33, or a modulator of said polypeptide, such that the psychiatric disorder is treated.

50. The method of claim 49, wherein the psychiatric disorder is schizophrenia.

51. The method of claim 49, wherein the psychiatric disorder is schizoaffective disorder.

52. The method of claim 49, wherein the psychiatric disorder is bipolar affective disorder.

53. The method of claim 49, wherein the psychiatric disorder is unipolar affective disorder.

54. The method of claim 49, wherein the psychiatric disorder is adolescent conduct disorder.

55. The method of claim 44, wherein the modulator is selected from the group consisting of a mGluR5M nucleic acid molecule, a mGluR5M antibody or active antibody fragment, a ribozyme, an antisense nucleic acid molecule, a small molecule modulator, a peptide and a peptidomimetic.

56. The method of claim 45, wherein the modulator is selected from the group consisting of a mGluR5M nucleic acid molecule, a mGluR5M antibody or active antibody fragment, a ribozyme, an antisense nucleic acid molecule, a small molecule modulator, a peptide and a peptidomimetic.

57. The method of claim 48, wherein the modulator is selected from the group consisting of a mGluR5M nucleic acid molecule, a mGluR5M antibody or active antibody fragment, a ribozyme, an antisense nucleic acid molecule, a small molecule modulator, a peptide and a peptidomimetic.

58. The method of claim 49, wherein the modulator is selected from the group consisting of a mGluR5M nucleic acid molecule, a mGluR5M antibody or active antibody fragment, a ribozyme, an antisense nucleic acid molecule, a small molecule modulator, a peptide and a peptidomimetic.
